(19) United States
(12) Patent Application Publication
(10) Pub. No.: US 2017/0367989 A1
(43) Pub. Date: Dec. 28, 2017

(54) NANOPARTICLES FOR LIPID HOMEOSTASIS

(71) Applicants: Shanta DHAR, Miami, FL (US); Sean MARRACHE, Portland, OR (US); Ru WEN, Athens, GA (US); Bhabatosh BANIK, Athens, GA (US); UNIVERSITY OF GEORGIA RESEARCH FOUNDATION, INC., Athens, GA (US)

(72) Inventors: Shanta Dhar, Miami, FL (US); Sean Marrrache, Portland, OR (US); Ru Wen, Athens, GA (US); Bhabatosh Banik, Miami, FL (US)

(21) Appl. No.: 15/536,832

(22) PCT Filed: Dec. 22, 2015
(86) PCT No.: PCT/US2015/067403

§ 371 Date: Jun. 16, 2017

Related U.S. Application Data
(60) Provisional application No. 62/095,599, filed on Dec. 22, 2014.
(60) Provisional application No. 62/099,996, filed on Jan. 5, 2015.

(51) Int. Cl. A61K 9/51; A61K 49/18; A61K 9/28; A61K 47/69; A61K 9/127; A61K 47/54; B82Y 5/00; A61K 45/06

(52) U.S. Cl. CPC A61K 9/5153; B82Y 5/00; A61K 49/1824; A61K 45/06; A61K 47/6937; A61K 9/1275; A61K 47/549; A61K 9/282

(57) ABSTRACT

Nanoparticles include a polymeric core and a high density lipoprotein (HDL) component where the ratio by weight of the HDL component to the polymeric core is in a range from about 1:9 to about 9:1, such as about 75:25 or less or about 7:3 or less. The nanoparticles may also include a mitochondria targeting moiety. The nanoparticles may be used to treat or prevent atherosclerosis or to maintain lipid homeostasis.

### RELATED APPLICATIONS

This application claims the benefit of U.S. Provisional Patent Application No. 62/095,599, filed on Dec. 22, 2014 and U.S. Provisional Patent Application No. 62/099,996, filed on Jan. 5, 2015, which provisional patent applications are hereby incorporated herein by reference in their respective entireties to the extent that they do not conflict with the disclosure presented herein.

### STATEMENT OF GOVERNMENTAL SUPPORT

This invention was made with government support under grant number R56HL121392, awarded by the National Heart, Lung and Blood Institute of the NIH of the United States government. The government has certain rights in the invention.

### STATEMENT OF ADDITIONAL SUPPORT

The work presented herein was also supported by an American Heart Association National Scientist Award 14SDG18690009.

### FIELD

The present disclosure relates to therapeutic nanoparticles, particularly to cholesterol mimicking nanoparticles, and methods of use thereof.

### SUMMARY

In various embodiments describe herein, a nanoparticle includes a polymeric core and a high density lipoprotein (HDL) component where the ratio by weight of the HDL component to the polymeric core is in a range from about 1:9 to about 9:1. Preferably, the weight ratio of the HDL component to the polymeric core is about 75:25 or less such as about 7:3 or less. That is, the HDL component constitutes about 75% or less, such as about 70% or less, of the combined weight of the HDL component and the polymeric core. The nanoparticle preferably also includes a mitochondria targeting moiety.

As described herein, various embodiments of nanoparticles having a polymeric core and an low relative amount of an HDL component (such as 10% or 20% HDL component relative to the total weight of the polymeric core and the HDL component) demonstrated higher cholesterol binding than nanoparticles having a high relative amount of the HDL component (such as 80% or 90%). Similarly, the cholesterol binding constant (k_{d}) was higher for lower relative amounts of HDL component than it was for higher relative amounts of HDL component. Nanoparticles having a polymeric core and an HDL component as described herein were demonstrated to have cholesterol binding constants in the low micromolar range, which is substantially higher than gold-core based HDL nanoparticles which previously have been shown to have cholesterol binding constants in the lower nanomolar range. Bound cholesterol was released more slowly from nanoparticles having lower amounts of HDL component relative to those having higher amounts of HDL components, suggesting that release kinetics can be varied by controlling the relative amount of HDL component incorporated into the nanoparticle.

Surprisingly, nanoparticles having mitochondria targeting moieties were found to bind substantially more cholesterol in in vitro studies than corresponding nanoparticles having corresponding amounts of HDL component but no mitochondria targeting moiety.

Various embodiments of nanoparticles described herein are shown to effectively reduce lipid levels in macrophages when used in a preventative manner or when used in a therapeutic manner. This is surprising because native HDL less effective when used in a preventative manner. As described in more detail below, nanoparticles having lower relative amounts of HDL component, such as nanoparticles having about 40% HDL component (relative to the total weight of the polymer core and the HDL component) are shown herein to reduce lipid levels in macrophages when used in both a preventative and a therapeutic manner. Nanoparticles having higher relative amounts of HDL component, such as nanoparticles having about 70% HDL component (relative to the total weight of the polymer core and the HDL component) are shown herein to more effectively reduce lipid levels in macrophages when used in a preventative manner than when used in a therapeutic manner. These results suggest that the effective use of nanoparticles as described here can be modified by varying the HDL content of the nanoparticles. In addition or alternatively, nanoparticles having differing concentrations of HDL component may be used in combination to achieve desired preventative or therapeutic effects.

Nanoparticles having both low and high relative amounts of HDL component were able to reduce differentiation of macrophages into foam cells when used in a preventative or a therapeutic manner, while native HDL was only able to prevent such differentiation when used in a therapeutic manner.

Further, nanoparticles having different relative HDL component concentration were shown to having different biodistribution profiles. Accordingly, the HDL component concentration of nanoparticles as described herein may be varied as appropriate to target desired tissue for treatment or prevention. In addition or alternatively, nanoparticles having differing concentrations of HDL component may be used in combination to achieve desired preventative or therapeutic effects.

### BRIEF DESCRIPTION OF THE DRAWINGS

FIG. 1. (Panel A) Schematic representation of the possible structural orientation of T-HDL-NPs. (Panel B) Various components and their structure used in the construction of synthetic T-HDL-NPs. (Panel C) A cartoon demonstrating variation in the components to generate library of T-HDL-NPs. (Panel D) Percent loading and % EE of CO in T-HDL-NP library as determined by the Amplex Red assay.

FIG. 2. Characterization of libraries of T and NT-HDL NPs. (Panel A) Top: Hydrodynamic diameter, zeta potential, and PDI of T-HDL-NPs with varied PLGA-COOH:CO. Bottom: TEM of 40% and 70% CO feed T-HDL-NPs. (Panel B) Top: Hydrodynamic diameter, zeta potential, and PDI of NT-HDL-NPs with varied PLGA-COOH:CO. Bottom: TEM of 40% and 70% CO feed NT-HDL-NPs.

FIG. 3. (Panel A) Comparison of time dependent NBD-cholesterol binding profiles of the T-HDLNP library with varied % CO feed. RFU: Relative fluorescence unit. (Panel B) Comparison of NBDcholesterol binding constants at 6 h of the T-HDL-NP library. (Panel C) Time dependent NBDcholesterol binding profiles of selected formulation of NT-HDL-NPs and hHDL. Comparison of NBD-cholesterol binding constants at 6 h of selected formulations of T-HDL-NP, NT-HDLNPs, and hHDL. (Panel D) Release of bound NBD-cholesterol from T-CO40-HDL-NPs and T-CO70-HDL-NPs under physiological pH of 7.4 at 37° C.

FIG. 4. Experimental design to study RCT mimicking properties of T-CO40-HDL-NPs and TCO70-HDL-NPs using macrophage derived foam cells under preventive and therapeutic settings.

FIG. 5. Lipid reduction by T-CO40-HDL-NPs and T-CO70-HDL-NPs in foam cells under therapeutic and preventive settings using confocal microscopy. Intracellular lipids were stained using AdipoRed and live cell imaging was performed. Representative AdipoRed staining (red) showed formation of foam cells from RAW 264.7 macrophages on treatment with oxLDL. Treatment with hHDL, T-CO40-HDL-NPs, and T-CO70-HDL-NPs were carried out before addition of oxLDL under preventive settings and after treatment with oxLDL under the therapeutic settings. Scale bar: 25 μm.

FIG. 6. Formation of foam cells from RAW 264.7 macrophages on treatment with oxLDL. Treatment with hHDL, T-CO40-HDL-NPs, and T-CO70-HDLNPs were carried out before addition of oxLDL under preventive settings and after treatment with oxLDL under the therapeutic settings. (Panel A) Lipid reduction by T-CO40-HDL-NPs and T-CO70-HDL-NPs in foam cells under therapeutic and preventive settings by AdipoRed assay. Intracellular triglyceride contents were stained using AdipoRed and quantified using a plate reader. (Panel B) Increased ROS levels after foam cell formation and subsequent reduction in oxidative stress level in foam cells after treatment with T-CO40-HDL-NPs or T-CO70-HDL-NPs or hHDL. *, **, ***P value 0.01-0.05, 0.001-0.01, and <0.001, respectively. ns: non significant.

FIG. 7. (Panel A) Accumulation of T-CO40-HDL-NPs in the aorta and T-CO70-HDL-NPs in the heart of normal pigs. American Landrace piglets were anesthetized and T-CO40-HDL-NPs or T-CO70-HDL-NPs (1.25 mg/kg with respect to NP) were administered. NPs were quantified by ICP-MS and IVIS analyses of plasma and organ samples to determine the PK parameters and bioD profiles. P value <0.001 for ***; ns: non-significant. (Panel B) Levels of plasma total cholesterol and triglyceride in piglets administered with T-CO40-HDL-NPs and T-CO70-HDL-NPs. *, **, ***P value 0.01-0.05, 0.001-0.01, and <0.001, respectively. ns: non-significant. (Panel C) Cytokine signals from pig plasma samples for analyses of anti-inflammatory properties of T-CO40-HDL-NPs and T-CO70-HDL-NPs in piglets. **, ***P value 0.001-0.01 and <0.001, respectively.

FIG. S1. Overlay of DLS plots of (Panel A) diameter and (Panel B) zeta potential of T-HDL-NPs.

FIG. S2. Overlay of DLS plots of (Panel A) diameter, (Panel B) zeta potential, (Panel C) percent loading of CO, and (Panel D) percent encapsulation efficiency (EE) of CO of NT-HDL-NPs.

FIG. S3. TEM images of library of T-HDL-NPs.

FIG. S4. TEM images of library of NT-HDL-NPs.

FIG. S5. Comparison of time dependent NBD-cholesterol binding profiles of the NTHDL-NP (0.025 mg/mL) library with varied % CO feed. RFU: Relative fluorescence unit.

FIG. S6. Comparison of NBD-cholesterol binding constants at 6 h of the NT-HDL-NP library.

FIG. S7. Accumulation of T-CO40-HDL-NPs in the aorta and T-CO70-HDL-NPs in the heart of normal pigs in units of % ID/g tissue. American Landrace piglets were anesthetized and T-CO40-HDL-NPs or T-CO70-HDL-NPs (1.25 mg/kg with respect to NP) were administered. NPs were quantified by ICP-MS.

FIG. S8. Heart and aorta images from all animals. American Landrace piglets (4 weeks old) were anesthetized using isofluorane and T-CO40-HDL-NPs were administered via ear vein IV. Distribution of NPs was studied by performing IVIS analyses of the whole heart and aorta. The data show the images of all the animals from each group.

FIG. S9. Heart and aorta images from all animals. American Landrace piglets (4 weeks old) were anesthetized using isofluorane and T-CO70-HDL-NPs were administered via ear vein IV. Distribution of NPs was studied by performing IVIS analyses of the whole heart and aorta. The data show the images of all the animals from each group.

FIGS. S10A-E. (A) Therapeutic efficacy of T-CO₄₀-HDL-NP and NT-CO₄₀-HDL-NP in apoE^{-/-} mouse model fed with normal diet. Animals were treated T-CO₄₀-HDL-NPs or NT-CO₄₀-HDL-NPs at a dose of 10 mg/kg (with respect to total NP) twice weekly via intravenous injection. Plasma lipid levels were compared between the treatments by quantifying triglyceride, total cholesterol, LDL, and HDL. (B) Oil red 0 staining of aortic valves from saline, T-CO₄₀-HDL-NP, and NT-CO₄₀-HDL-NP treated apoE^{-/-} animals. (C) Amount of cleaved caspase-3-positive areas from aorta and myocardium from saline, T-CO₄₀-HDL-NP, and NT-CO₄₀-HDL-NP treated apoE^{-/-} animals (D) H and E stained aorta and aortic valve from T-CO₄₀-HDL-NP and NT-CO₄₀-HDL-NP treated apoE^{-/-} animals. (E) H and E stained images of all major organs from T-CO₄₀-HDL-NP and NT-CO₄₀-HDL-NP treated apoE^{-/-} animals demonstrating no significant toxicity.

Schematic drawings presented herein are not necessarily to scale.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

Atherosclerosis is one of the most common causes of death in the western world. At the cellular level, dysfunctional cholesterol homeostasis in macrophages leading to foam cells contributes to atherosclerotic plaque formation within the intimal layers of the arteries. Macrophage derived foam cells loaded with high levels of cholesterol and cholesteryl ester (CE) directly influence plaque stability and progression. The extent of cholesterol removal from foam cells by cellular cholesterol efflux is an orchestrated pathway operated by ATP-binding cassette (ABC) transporters, apolipoproteins such as apoA-I, apoE, and high-density lipoproteins (HDL). The extracellular cholesterol efflux by ABC transporters, apoA-I and HDL is well studied.

However, efficient removal of cholesterol from foam cells depends on the intracellular lipid transport and is not well understood. The intracellular cholesterol transport regulates the sterol contents in organelles such as mitochondria, lipid rafts and membranes, and influence formation of cytosolic CE droplets. Steroidogenic acute regulatory protein (StAR)-related lipid transfer (START) domain with a hydrophobic lipid binding pocket participate in maintenance of cellular cholesterol homeostasis. Thus the relative contribution of extra- and intracellular cholesterol plays significant roles in removal of lipids from foam cells. However, these events are not well understood. Cholesterol serves as an obligatory precursor for steroid hormone production in steroidogenic tissues. Regardless of the tissue of origin or the steroid hormone to be produced, steroidogenesis is initiated by the cleavage of the cholesterol side chain to produce pregnenolone. The protein catalyzing this reaction, P-450 side chain cleavage enzyme (P450scc), is located in the inner mitochondrial membrane (IMM) facing the mitochondrial matrix. This enzyme activity is not the rate limiting in the process; instead, it is the delivery of cholesterol to the mitochondria by StAR, a cytosolic protein with a mitochondrial targeting signal.

Under conditions of atherosclerosis where cells are challenged with massive fluxes of cholesterol, efficiency of StAR protein decreases. Mitochondrial matrix contains less cholesterol since the outer mitochondrial membrane (OMM) contains more cholesterol than the IMM. Delivery of cholesterol to the IMM by StAR and activity of P450scc generates pregnenolone inside the mitochondria from cholesterol. Thus StAR transports cholesterol to mitochondrial sterol 27-hydroxylase (CYP27A1), generating oxysterol ligands for the liver X receptor (LXR), a member of the nuclear receptor family of transcription factors, resulting in reduction of macrophage lipid mass and inflammatory responses.

An inverse association between HDLs and the risk of coronary heart diseases (CHDs) suggests that therapies with the ability to raise HDL levels may lead to positive outcomes. HDLs have atheroprotective properties and major activities include: roles in the extra cellular reverse cholesterol transport (RCT) pathway, directly by removing cholesterol from foam cells and inhibiting the oxidation of low-density lipoproteins (LDLs), by limiting the inflammatory processes, demonstrating antithrombotic properties. Along with apo E, which promotes cholesterol efflux from foam cells, apoA-I containing HDL is thought to facilitate the transport of cholesterol from lesions. Thus a synthetic, biodegradable, mitochondria targeted HDL mimicking nanoparticle (NP) could provide benefits for managing excess cholesterol and maintenance of both extra and intracellular lipid homeostasis.

As the commercialization of nanotechnology continues to expand, the ability to translate particle fabrication methods from a laboratory to an industrial scale is of increasing significance. Therefore, development of a completely synthetic, scalable, biodegradable, and mitochondria-targeted HDL-mimicking NP can provide a unique therapeutic strategy for CHD. This HDL-mimicking NP will have the ability to decrease plaque inflammation directly by locally accumulating HDL mimic in plaque macrophages, working at the intracellular cholesterol metabolism pathways by targeting the mitochondria, and can further decrease mitochondrial ROS by taking advantage of HDL anti-oxidative properties. A mitochondria targeted HDL mimicking NP will be able to carry excess cholesterol from cytosol to the mitochondria to play an important role in the maintenance of extra and intracellular lipid homeostasis.

This disclosure describes, among other things, characterization, scale-up, and therapeutic potential of mitochondria targeted HDL mimics using, among other things, a piglet model. In various embodiments, described herein, an HDL-mimicking nanoparticle includes biocompatible polymers and lipids to create a synthetic yet biodegradable HDL mimic. The low cost small molecule based targeting strategies, as presented here, will provide evident advantages such as high specificity and reduced side effects. In the last few decades, reconstituted HDL (rHDL) has been investigated as a drug delivery system. The main limitation for the therapeutic application of rHDL is the necessity to isolate HDL apolipoproteins from human serum, or to resort to cell expression systems that secrete recombinant human apoA-I. In some embodiments, a completely synthetic, biodegradable, and mitochondria targeted HDL mimicking nanoparticle (NP) with the potential to participate in both extracellular and intracellular lipid homeostasis is described herein.

Nanoparticles described herein include a polymeric core and a high density lipoprotein (HDL) component, where the ratio by weight of the HDL component to the polymeric core is in a range from about 1:9 to about 9:1. Preferably, the weight ratio of the HDL component to the polymeric core is about 75:25 or less such as about 7:3 or less. That is, the HDL component constitutes about 75% or less, such as about 70% or less, of the combined weight of the HDL component and the polymeric core. The nanoparticles preferably also include a mitochondria targeting moiety and may optionally include one or more additional targeting moiety to target the nanoparticles to appropriate cells, such as macrophages, foam cells or the like. The targeting moiety preferably extends outwardly from the core to be available for interaction with cellular components, which interactions will target the nanoparticles to the appropriate cells, organelles, or the like. The targeting moieties may be tethered to the core or components that interact with the core.

Nanoparticles, as described herein, may include one or more contrast agents or one or more therapeutic agents in addition to the HDL component. In some embodiments, the contrast agents or therapeutic agents are contained or embedded within the core. If the nanoparticle includes therapeutic agents, the agents are preferably released from the core at a desired rate. In some embodiments, the core is biodegradable and releases the agents as the core is degraded or eroded.

I. Polymer Component

A nanoparticle as described herein may comprise one or more polymer. Preferably the nanoparticle comprises a core formed from at least one polymer. For purposes of the present disclosure, a core comprising at least one polymer is a “polymeric core.” A nanoparticle as described herein may also optionally include a polymeric layer or shell surrounding the polymeric core. In various embodiments, the polymeric core comprises a hydrophobic polymer or a hydrophobic portion of a polymer. In some embodiments, the shell comprises a hydrophilic polymer or a hydrophilic portion of a polymer. The hydrophobic portions can form the core, while the hydrophilic regions may for a shell that helps the nanoparticle evade recognition by the immune system and enhances circulation half-life. For example, a hydrophobic portion of an amphiphilic block copolymer may form the core or a portion of the core, and a hydrophilic portion of an amphiphilic block copolymer may form the shell or a portion of the shell. Such amphiphilic block copolymers have hydrophobic portions and hydrophilic portions that may self-assemble in an aqueous environment into particles having the hydrophobic core and a hydrophilic surface around the core.

Examples of amphiphilic polymers include block copolymers having a hydrophobic block and a hydrophilic block. In embodiments, the core is formed from hydrophobic portions of a block copolymer, a hydrophobic polymer, or combinations thereof.

In some embodiments, the core, the shell, if present, or the core and the shell, if present, comprise one or more biodegradable polymer or a polymer having a biodegradable portion.

Any suitable synthetic or natural bioabsorbable polymers may be used. Such polymers are recognizable and identifiable by one or ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles (e.g., of compounds contained in a polymeric matrix comprising the polymers) of these and other suitable polymers are known or readily identifiable.

In various embodiments, described herein the core comprises PLGA. PLGA is a well-known and well-studied hydrophobic biodegradable polymer used for the delivery and release of therapeutic agents at desired rates.

Non-limiting examples of polymers that may be used to form a core or a shell of a nanoparticle include one or more of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polyureas, polystyrenes, and/or polyamines. In some embodiments, the polymeric core comprises a polyester. In some embodiments, a polymeric matrix may comprise poly(lactic-co-glycolic acid) (PLGA), polyethylene glycol (PEG), and/or copolymers thereof.

Polymers may be natural or unnatural (synthetic) polymers. Polymers may be homopolymers or copolymers comprising two or more monomers. In terms of sequence, copolymers may be random, block, or comprise a combination of random and block sequences. Typically, a nanoparticle having a polymer component will comprise an organic polymer.

Examples of polymers include polyethylenes, polycarbonates (e.g. poly(1,3-dioxan-2one)), polyanhydrides (e.g. poly(sebacic anhydride)), polyhydroxyacids (e.g. poly(β-hydroxyalkanoate)), polypropylfumerates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines. In some embodiments, polymers in accordance with the present invention include polymers which have been approved for use in humans by the U.S. Food and Drug Administration (FDA) under 21 C.F.R. §177.2600, including but not limited to polyesters (e.g. polylactic acid, poly(lactic-co-glycolic acid), polycaprolactone, polyvalerolactone, poly(1,3-dioxan-2one)); polyanhydrides (e.g. poly(sebacic anhydride)); polyethers (e.g., polyethylene glycol); Polyurethanes; polymethacrylates; polyacrylates; and polycyanoacrylates. In some embodiments, polymers can be hydrophilic. For example, polymers may comprise anionic groups (e.g. phosphate group, sulphate group, carboxylate group); cationic groups (e.g. quaternary amine group); or polar groups (e.g. hydroxyl group, thiol group, amine group).

In some embodiments, polymers may be modified with one or more moieties, one or more functional groups, or one or more moieties and one or more functional groups. Any suitable moiety or functional group can be used. In some embodiments, polymers may be modified with polyethylene glycol (PEG), with a carbohydrate, with acyclic polyacetals derived from polysaccharides, or the like. In some embodiments, the polymer is modified with PEG.

In some embodiments, a polymer may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively. referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g. PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly [α-(4-aminobutyl)-L-gly colic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, a polymer may be a carbohydrate. In some embodiments, a carbohydrate may be a polysaccharide comprising simple sugars (or their derivatives) connected by glycosidic bonds, as known in the art. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan.

In some embodiments, a polymer may be a protein or peptide, properties of which are described in further detail below. Exemplary proteins that may be used in accordance with the present invention include, but are not limited to, albumin, collagen, gelatin, hemoglobin, a poly(amino acid) (e.g. polylysine), an antibody, etc.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step.

It is further to be understood that a polymer may include block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

One promising example of polymer-based nanomaterials is represented by a class of Pluronic® block copolymers (also known under non-proprietary name “poloxamers”). These block copolymers consist of hydrophilic poly(ethylene oxide) (PEO) and hydrophobic poly(propylene oxide) (PPO) blocks arranged in A-B-A tri-block structure: PEO-PPO-PEO

Any suitable hydrophilic polymer may form a hydrophilic block of a block copolymer. Examples of suitable hydrophilic polymers include polysaccharides, dextran, chitosan, hyaluronic acid, hydroxypropylmethylcellulose (HPMC), 2-hydroxymethacrylate and the like. In embodiments, polyethylene glycol (PEG) is a hydrophilic polymer used to serve as the hydrophilic portion of a block copolymer.

In some embodiments, the core comprises one or more lipids or lipid portions of molecules such as phospholipids. Phospholipids may form micelles having a hydrophobic core and a hydrophilic outer surface.

II. Targeting Moieties

The nanoparticles described herein include one or more moieties that target the nanoparticles to a desired cell-type, organelle, or the like. The targeting moieties may be tethered to the core in any suitable manner, such as binding to a molecule that forms part of the core or to a molecule that is bound to the core.

In embodiments, a targeting moiety is bound to a polymer that forms, or is bound to a polymer that forms, part of the core or that is bound to a lipid, such as a phospholipid, that is bound to, or forms part of, the core. In embodiments, a targeting moiety is bound to a hydrophilic portion of a block copolymer having a hydrophobic block that forms part of the core.

The polymers, or portions thereof, may contain, or be modified to contain, appropriate functional groups, such as —OH, —COOH, —NH₂, —SH, or the like, for reaction with and binding to the targeting moieties that have, or are modified to have, suitable functional groups for reacting and bonding with functional groups of the polymers.

Examples of targeting moieties tethered to polymers and lipids are presented throughout this disclosure for purpose of illustrating the types of reactions and tethering that may occur. However, one of skill in the art will understand that tethering of targeting moieties to polymers, lipids, polypeptides, or the like, may be carried out according to any of a number of known chemical reaction processes.

Targeting moieties may be present in the nanoparticles at any suitable concentration. In embodiments, the concentration may readily be varied based on initial in vitro analysis to optimize prior to in vivo study or use. In embodiments, the targeting moieties will have surface coverage of from about 10% to about 100%.

A nanoparticle described herein may include a mitochondria targeting moiety that facilitates accumulation of the nanoparticle in the mitochondrial matrix. Due to the substantial negative electrochemical potential maintained across the inner mitochondrial membrane [mitochondrial membrane potential (Δψm)], delocalized lipophilic cations are effective at crossing the hydrophobic membranes and accumulating in the mitochondrial matrix.

One suitable mitochondria-targeting delocalized lipophilic cation is a triphenylphosphonium (TPP) cation. TPP-containing compounds can accumulate greater than 1000 fold within the mitochondrial matrix. Any suitable TPP-containing compound may be used as a mitochondrial matrix targeting moiety. Representative examples of TPP-based moieties may have structures indicated below in Formula I, Formula II or Formula III:

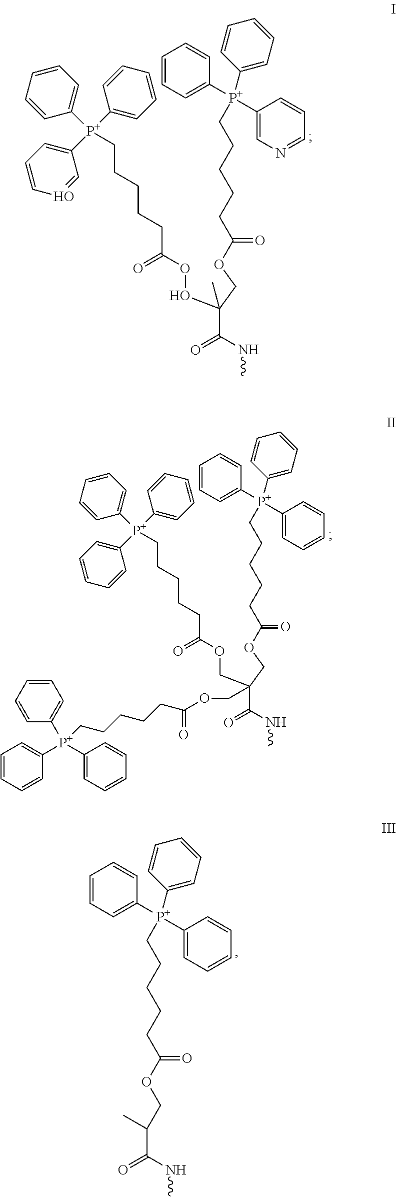

where the amine (as depicted) may be conjugated to a polymer, lipid, or the like for incorporation into the nanoparticle.

Another example of a compound having a TPP-based moiety that has a hydrophobic tail for incorporation into the core is show in Formula IV:

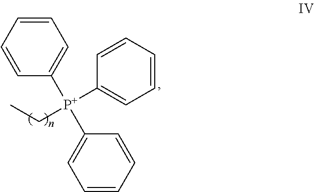

where n is an integer between 5 and 50, such as between 10 and 30 or between 15 and 20.

In embodiments, the delocalized lipophilic cation for targeting the mitochondrial matrix is a rhodamine cation, such as Rhodamine 123 having Formula V as depicted below:

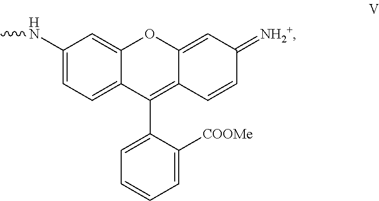

where the secondary amine (as depicted) may be conjugated to a polymer, lipid, or the like for incorporation into the nanoparticle. In addition or alternatively, a Rhodamine 123-containing compound can include a hydrophobic moiety, such as a long chain alkane, for incorporation into the core.

Of course, non-cationic compounds may serve to target and accumulate in the mitochondrial matrix. By way of example, Szeto-Shiller peptide may serve to target and accumulate a nanoparticle in the mitochondrial matrix. Any suitable Szetto-Shiller peptide may be employed as a mitochondrial matrix targeting moiety. Non-limiting examples of suitable Szeto-Shiller peptides include SS-02 and SS-31, having Formula IX and Formula X, respectively, as depicted below:

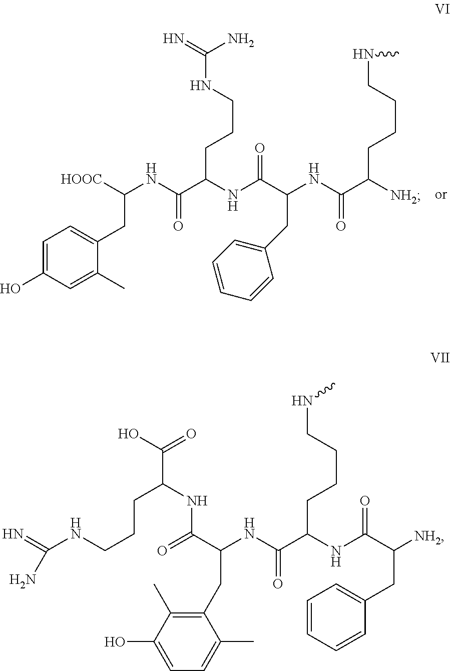

where the secondary amine (as depicted) may be conjugated to a polymer, lipid, or the like for incorporation into the nanoparticle. In addition or alternatively, a Szeto-Shiller peptide-containing compound can include a hydrophobic moiety, such as a long chain alkane, for incorporation into the core.

For purposes of example, a reaction scheme for synthesis of distearoyl-snglycero-3-phosphoethanolamine (DSPE)-PEG-TPP is shown below in Scheme I. It will be understood that other schemes may be employed to synthesize DSPE-PEG-TPP and that similar reaction schemes may be employed to tether other mitochondrial targeting moieties to DSPE-PEG or to tether moieties to other polymers, copolymers, or lipids.

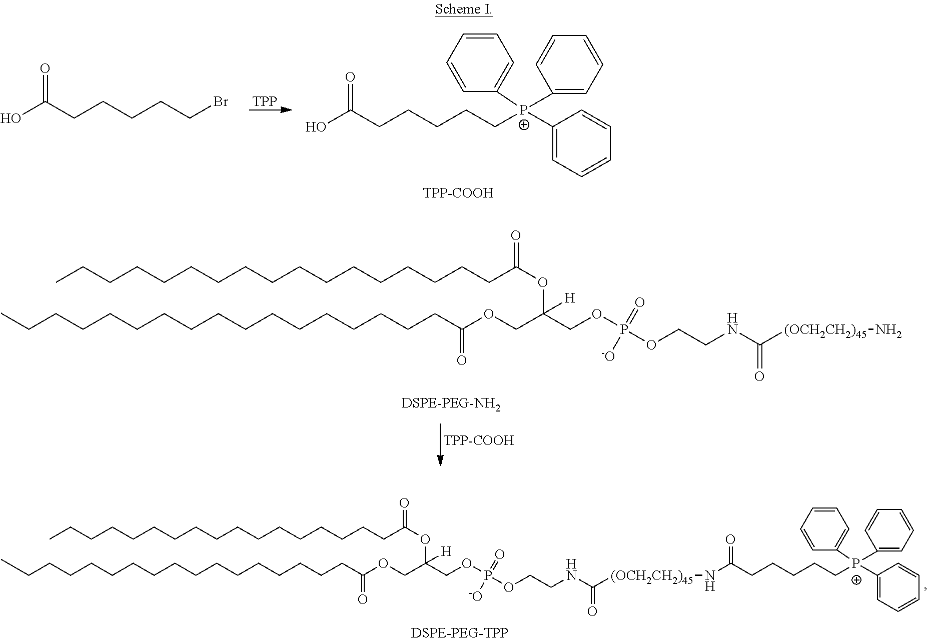

In some embodiments, a nanoparticle described herein includes a macrophage targeting moiety. Because macrophages are characteristically present in atherosclerosis lesions and/or because macrophages may be important in lipid homeostasis, it may be desirable to target nanoparticles described herein to macrophages.

Any suitable moiety may be used to increase the affinity of a nanoparticle for macrophage cells. For example, mannose or other simple sugar moieties may be incorporated into the nanoparticle as targeting moieties. Simple sugars, such as mannose and galactose, may selectively interact with macrophages through receptors. For example, macrophages contain macrophage mannose receptors and lectins that selectively bind galactose (macrophage galactose-binding lectin). Through these selective interactions, the presence of such simple sugars or compounds that include such simple sugars may be used to target the nanoparticles to macrophages. In embodiments, a macrophage targeting moiety includes mannose, galactose or lactobionic acid.

Because the presence of the simple sugars facilitates interactions of the nanoparticle with macrophages, the sugar moieties are preferable shielded in the nanoparticle until the nanoparticle reaches the desired location, such as an atherosclerotic plaque, to minimize exposure to the reticulo-endothelial system (RES).

The sugar moieties may be temporarily shielded in any suitable manner. In embodiments, the sugar moieties are shielded by a hydrophilic polymer, such as polyethylene glycol (PEG); a hydrophilic portion of a polymer, such as a block copolymer containing a PEG block; a hydrophilic polymer bound to a lipid that is bound to the core or is part of a compound that, at least in part, forms the core; or the like. The shielding polymer may form a portion of the core of the nanoparticle (e.g., when the polymer is a block co-polymer and a hydrophobic block of the polymer contributes to formation of the core) or may be otherwise bound to the core.

Preferably, the shielding polymer is releasable from the nanoparticle when the nanoparticle reaches its target location, such as an atherosclerotic plaque, to expose the macrophage-targeting moiety. For example, the shielding polymer may be bound to the core, a polymer, a lipid, or the like via a cleavable linker. The cleavable linker may be cleaved when reaching the target location. Any suitable cleavable linker may be employed. In embodiments, the cleavable linker is a peptide linker cleavable by a matrix metalloproteinase (MMP). One example of a peptide linker cleavable by MMP2 is a polypeptide having the amino acid sequence GPLGVRG (SEQ ID NO:2). Regardless of the linker employed, cleavage preferably takes place at cleavage enzyme (such as MMP) concentrations at or below those present in atherosclerotic plaques. In embodiments, cleavage of the cleavable linker induces surface switching that results in exposure of the macrophage-targeting moiety.

Other cleavable linkers that may be employed are those susceptible to cleavage by glutathione, pH changes, temperature changes, or the like.

Non-limiting examples of macrophage targeting moieties bound to polymers, lipids, or the like include mannose, galactose, or lactobionic acid bound to short hydrophilic polymer chains, such as PEG₅₀₀. In embodiments, the short hydrophilic polymer chains are bound to the core via a lipid or hydrophilic polymer. For example, PEG₅₀₀ may be bound to a hydrophobic polymer, such as PLGA, which forms at part of the core. In embodiments, the short hydrophilic polymers are bound to a lipid, such as distearoyl-snglycero-3-phosphoethanolamine (DSPE), which is bound to the core or to a hydrophilic polymer forming part of the core. Larger hydrophilic chains, such as PEG₃₄₀₀, may serve as shielding polymers. The shielding polymers may be bound to the core via a hydrophobic polymer, lipid or the like. The shielding polymers may be bound to the hydrophobic polymer, lipid, or the like via a cleavable linker.

In embodiments, the macrophage targeting moieties are bound to PEG, such as PEG₅₀₀, which is bound to PLGA (i.e., PLGA-PEG-targeting moiety). In embodiments, the macrophage targeting moieties are bound to PEG, such as PEG₅₀₀, which is bound to DSPE (i.e., DSPE-PEG-targeting moiety).

In embodiments the shielding polymer, such as PEG₃₄₀₀, is bound to PLGA (i.e., PLGA-PEG) and may be bound to PLGA via a cleavable linker (i.e., PLGA-linker-PEG). In embodiments, the shielding polymer, such as PEG₃₄₀₀, is bound to DSPE (i.e., DSPE-PEG) and may be bound to DSPE via a cleavable linker (i.e., DSPE-linker-PEG).

The macrophage-targeting moieties may be bound to the polymers or lipids in any suitable manner. By way of example, DSPE-PEG-mannose may be synthesized through amide coupling of D-mannosamine hydrochloride with DSPE-PEG-COOH; e.g., as shown in the following reaction scheme:

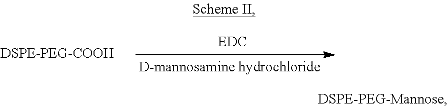

where EDC is 1-ethyl-3-(3-(dimethylamino)-propyl)carbodiimide.

DSPE-PEG-lactobionic acid may be synthesized according to the following reaction scheme:

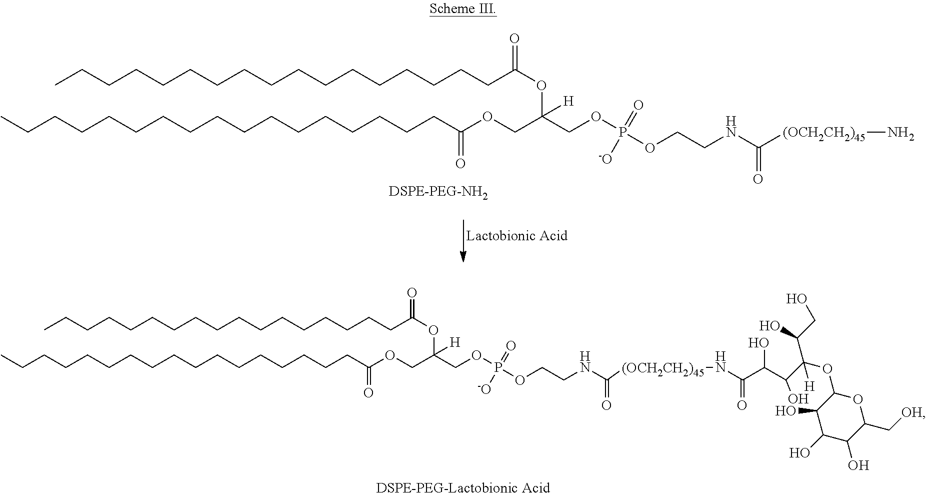

It will be understood that the reaction schemes shown in Schemes II and III are for purposes of illustration and that other reaction schemes may be employed, as well as other phospholipids, polymers, or mannose or galactose containing compounds.

Another example of a suitable tethered targeting moiety is a mannosyl alkyltriazole. A reaction scheme for synthesizing such a tethered targeting moiety is depicted below in reaction Schemes IV-VII.

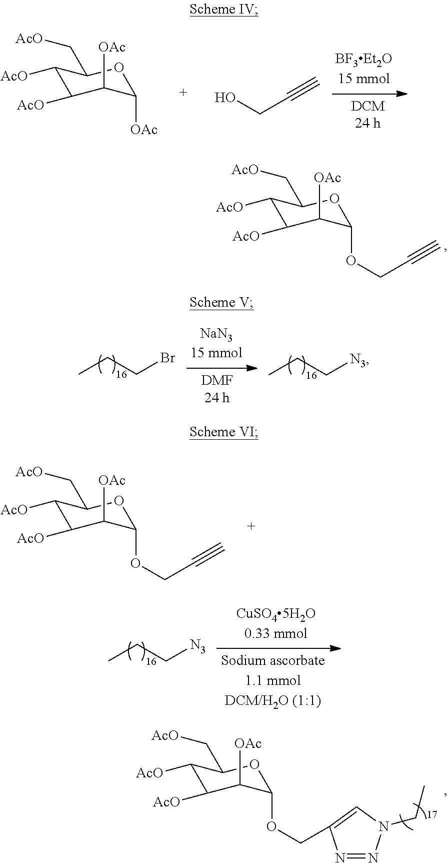

and

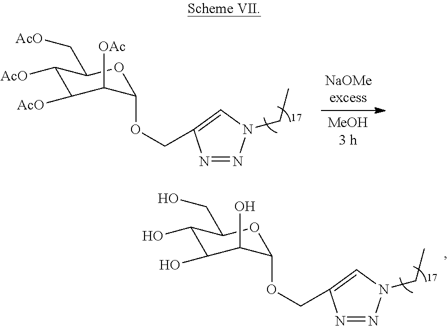

Schemes IV-VII may be modified to alter the number of carbons in the alkyl chain, to include a branched chain alkyl, or the like.

The alkyl chain may be embedded in, for example, a lipid layer of a nanoparticle to retain the tethered targeting moiety in the nanoparticle.

It will be understood that any other desirable and suitable targeting moiety, such as a foam cell targeting moiety, for example, may be incorporated into a nanoparticle described herein. It will also be understood that reactions schemes similar to those described above, or other suitable reaction schemes can be employed to tether a targeting moiety to a component suitable for incorporation into a nanoparticle.

III. Phospholipid Monolayer

In embodiments the nanoparticle includes a lipid or phospholipid monolayer, which may be present at the interface of the hydrophobic core of the nanoparticle and the hydrophilic shell. The phospholipids of the monolayer have hydrophobic tails that interact with the hydrophobic core of the nanoparticle. The phospholipids may thus be used to tether various components, such as targeting moieties or contrast agents, to the core. The lipid or phospholipid layer may also serve to prevent agents in the core from freely diffusing out of the core and may reduce water penetration into the core.

Any suitable lipid or phospholipid may be employed. Examples of lipids or phospholipids that may be employed include lecithin, distearoyl-snglycero-3-phosphoethanolamine (DSPE) of different chain lengths, cardiolipin, DSPE containing branched PEG, or the like, or mixtures thereof. Preferably, the lipids are biodegradable. In embodiments, DSPE, cardiolipin, or a mixture of DSPE and cardiolipin are used to form a phospholipid monolayer.

As discussed above, the lipids may be used to tether various components to the core. In embodiments, the tethered components are further tethered by intervening polymers, preferably hydrophilic polymers such as PEG. The lipids may include reactive groups, or may be modified to contain reactive groups, for reaction with and binding to various polymers (which have or may be modified to have appropriate reaction groups) or other components (which have or may be modified to have appropriate reaction groups).

Examples of lipid-based tethered molecules include DSPE-PEG-TPP discussed above. It will be understood that other phospholipids and hydrophilic polymers (other than DSPE and PEG) may be employed. It will be further understood that moieties may be tethered to a phospholipid, with or without an in intervening hydrophilic polymer, in a similar manner.

IV. HDL Components

In embodiments, the nanoparticle includes HDL or HDL-mimicking components, which will be collectively referred to herein as “HDL components.” HDLs may oppose atherosclerosis directly, by removing cholesterol from foam cells, by inhibiting the oxidation of low-density lipoproteins (LDLs), and by limiting the inflammatory processes that underlie atherosclerosis. HDLs may also have anti-thrombogenic properties. Thus, HDL-cholesterol (HDL-C) may interrupt the process of atherogenesis at several key stages. Along with apoE, which promotes cholesterol efflux from foam cells, apoA-1 containing HDL is thought to facilitate the transport of cholesterol from lesions. Accordingly, incorporating HDL components and associated components, such as apoA-1 protein or a synthetic mimetic thereof, into a nanoparticle that targets atherosclerotic lesions may provide significant benefits to patients.

In embodiments, an HDL component incorporated into the nanoparticle comprises a cholesterol, such as cholesteryl oleate, reconstituted HDL from human plasma, or the like. In embodiments, the cholesterol is incorporated into, or forms a part of, the hydrophobic core of the nanoparticle. In embodiments, the cholesterol may incorporate into phospholipid monolayer, if present in the nanoparticle.

In various embodiments, the ratio of the HDL component to the polymer(s) forming the core of a nanoparticle are tailored to achieve a desired result, such as maintenance of lipid homeostasis. In some embodiments, the ratio of HDL component to core polymer component is in a range from about 1:9 to about 9:1. Preferably, the weight ratio of the HDL component to the polymeric core is about 75:25 or less such as about 7:3 or less. That is, the HDL component constitutes about 75% or less, such as about 70% or less, of the combined weight of the HDL component and the polymeric core. In some embodiments, the ratio of HDL component to core polymer component is in a range from about 1:4 to about 3:2. In some embodiments, the ratio of HDL component to core polymer component is 2:3. For purposes of the present disclosure, weight ration of the HDL component to the core polymer component or “polymeric core” refers to the weight ratio of the HDL component to the polymeric portion of the core. Accordingly, if the core comprises components in addition to polymeric components the weight of the additional components is not taken into account for purposes of calculating the HDL component:polymeric core weight ratio. In addition, if a polymer includes a first portion that forms at least a portion of the core and another portion that forms another part of the nanoparticle, such as a shell surrounding the core, the weight of only that portion of the polymer that forms the core is taken into account for determining the HDL component:polymeric core weight ratio for purposes of this disclosure.

In some embodiments, the HDL component comprises cholesteryl oleate and the core polymer comprises PLGA, such as PLGA-COOH. In some embodiments, the HDL component consists essentially of cholesteryl oleate and the core polymer consists essentially of PLGA, such as PLGA-COOH

ApoA-1 or any suitable apoA-1 peptide mimetic may be incorporated into the nanoparticle. One example of an apoA-1 peptide mimetic is a polypeptide having the amino acid sequence FAEKFKEAVKDYFAKFWD. (SEQ ID NO:1). ApoA-1 or apoA-1 mimetics will tend to self-assemble into the nanoparticles, particularly if the nanoparticle includes a phospholipid monolayer, and thus need not be tethered to other components such as polymers or lipids. However, such polypeptides may be tethered to polymers or lipids.

In embodiments, a nanoparticle includes self-assembled apoA-1 peptide network and a colloidal phospholipid monolayer mimic plasma derived HDL.

V. Contrast Agents

A nanoparticle (NP) as described herein may include one or more contrast agents for purpose of imaging, visualization or diagnosis. Any suitable contrast agent may be employed. In embodiments, the contrast agent is suitable for in vivo magnetic resonance imaging (MRI), such as iron oxide (TO) nanocrystals. In embodiments, the contrast agent is suitable for ex vivo/in vivo optical imaging, such as quantum dot (QD) (fluorescence), cdots, pdots, or the like. In embodiments, the nanoparticle includes both contrast agents for MRI and agents for fluorescent optical imaging.

A single construct containing complementary imaging agents could be of enormous benefits for atherosclerosis. NPs with the ability to carry both fluorescent (QD) and MRI (TO) probes represent an unique platform, which can find wide preclinical applications in the study of inflammatory atherosclerosis, postinfarction healing, transplant rejection, and early aortic valve disease, to name a few. These contrast agents enable the attainment of both high imaging sensitivity from fluorescence and high spatial resolution from MRI, which also helps to compensate for the limited imaging depths of fluorescence imaging. A targeted single NP platform containing both fluorescence and MRI contrast agents to allow imaging of apoptotic macrophages in the vulnerable plaque could help open the door for many promising human applications, including imaging of micro thrombi associated with vulnerable coronary plaques.

As both QD and IO can be synthesized in the same size range and with similar capping ligands, the NP platform will be uniform and can allow facile exchange of the components without significantly altering the overall properties of the NP. The localized regions of higher fluorescence signal seen ex vivo using this platform may be useful for correlating with vulnerable plaques identified in vivo by MRI.

Contrast agents may be incorporated into the NP in any suitable manner. In embodiments, the contrast agents are incorporated into the core or are contained within the core. In embodiments, the contrast agents are tethered to a lipid, polymer, protein or other component of the nanoparticle. Such tethering can be carried out as described above with regard to other components of the nanoparticle, such as targeting moieties.

By way of example, as described herein QD has been conjugated to PEG to form QD-conjugated amine-terminated PEG (NH₂-PEG-QD) that was conjugated to PLGA-COOH to produce PLGA-b-PEG-QD.

Contrast agents may be present in a nanoparticle in any suitable amount. In embodiments, a contrast agent is present in a nanoparticle from about 0.05% by weight to about 20% by weight of the nanoparticle.

VI. Therapeutic Agents

In some embodiments, the nanoparticle has no separate therapeutic agent and the nanoparticle itself, with and HDL component and optionally and ApoA-I mimetic component, serves as a therapeutic agent.

However, a nanoparticle, as described herein, may include any one or more therapeutic agents. The therapeutic agent may be embedded in, or contained within, the core of the nanoparticle. Preferably, the therapeutic agent is released from the core at a desired rate. If the core is formed from a well-known and well-studied polymer (such as PLGA) or combination of polymers, the release rate can be readily controlled.

In embodiments, a therapeutic agent or precursor thereof is conjugated to a polymer, lipid, etc., in a manner described above with regard to targeting moieties. The therapeutic agent may be conjugated via a cleavable linker so that the agent may be released when the nanoparticle reaches the target location, such as an apoptotic macrophage.

The therapeutic agents may be present in the nanoparticle at any suitable concentration. For example, a therapeutic agent may be present in the nanoparticle at a concentration from about 0.01% to about 20% by weight of the nanoparticle.

In embodiments, the nanoparticle includes one or more therapeutic agent useful for treatment of vascular plaques or atherosclerosis, or for maintaining lipid homeostasis. For example, a nanoparticle may include one or more statin, one or more fibrate, or combinations thereof. Suitable statins include atorvastin, simvastatin, and lovastatin. Suitable fibrates include bezafibrate, fenofibrate, and gemfibrizol. In embodiments, atorvastatin is present in combination with one or more of simvastatin, lovastatin, bezafibrate, fenofibrate, or gemfibrizol.

In some embodiments, a nanoparticle includes one or more antioxidants, such as α-tocopherol acetate, glutathione, lipoic acid, uric acid, ascorbic acid, butylatedhydroxytoluene, d-α-tocopherol, monothioglycerol, sodium bisulfite, sodium sulfite, tocopherols, acetone sodium bisulfite, ascorbyl palmitate, cysteine, nordihydruguaiaretic acid, sodium formaldehyde sulfoxylate, sodium thiosulfate, acetylcysteine, butylated hydroxyanisole, cysteine hydrochloride, dithiothreitol, propyl gallate, sodium metabisulfite, thiourea, and the like. In some embodiments, a nanoparticle includes CoQ10.

In embodiments, a nanoparticle includes chenodeoxylcholic acid or another repressor (e.g., siRNA) of PCSK9. Repression of PCSK9 promotes hepatic LDL receptor degradation, and may enhance efficacy of statins or fibrates when used in combination.

VII. Size of Nanoparticle

Nanoparticles, as described herein, may be of any suitable size. Generally, the nanoparticles are of a diametric dimension of less than about 999 nanometers, such as less than about 750 nm, less than about 600 nm, less than about 500 nm, less than about 400 nm, less than about 300 nm, or less than about 200 nm. In addition, or alternatively, the nanoparticles may be of a diametric dimension of greater than about 5 nm. In embodiments, the nanoparticles are from about 30 nm to about 300 nm in diameter. In embodiments, the nanoparticles are separated according to size, such as from about 20 nm to about 40 nm, from about 40 nm to about 60 nm, from about 60 nm to about 80 nm, from about 80 nm to about 100 nm, or from about 100 nm to about 150 nm. In some embodiments, the average size of the nanoparticles is in a range from about 50 nm to about 200 nm, such as from about 75 nm to about 150 nm.

The size of the nanoparticle is one factor that may influence bio-distribution and mitochondrial uptake, if appropriate.

VIII. Zeta Potential

Nanoparticles as described herein can have any suitable zeta potential. Zeta potential is a term for electro kinetic potential in colloidal systems. While zeta potential is not directly measurable, it can be experimentally determined using electrophoretic mobility, dynamic electrophoretic mobility, or the like. Zeta potential can play a role in the ability of nanoparticles to accumulate in mitochondria, with higher zeta potentials generally resulting in increased accumulation in the mitochondria.

In some embodiments, the nanoparticles described herein have a zeta potential of greater than 0 mV, such as greater than 10 mV, greater than 20 mV, greater than 30 mV, or greater than 40 mV. A nanoparticle can have a zeta potential of less than 100 mV. In some embodiments, a nanoparticle has a zeta potential in a range from about 0 mV to about 60 mV, such as from about 10 mV to about 50 mV. In some embodiments, a nanoparticle has a zeta potential of about 40 mV.

Any suitable moiety that may be charged under physiological conditions may be a part of or attached to a hydrophilic polymer or hydrophilic portion of a polymer. In embodiments, the moiety is present at a terminal end of the polymer or hydrophilic portion of the polymer. Of course, the moiety may be directly or indirectly bound to the polymer backbone at a location other than at a terminal end. Due to the substantial negative electrochemical potential maintained across the inner mitochondrial membrane, cations, particularly if delocalized, are effective at crossing the hydrophobic membranes and accumulating in the mitochondrial matrix. Cationic moieties that are known to facilitate mitochondrial targeting are discussed in more detail above. However, cationic moieties that are not particularly effective for selective mitochondrial targeting may be included in nanoparticles or be bound to hydrophilic polymers or portions of polymers. In embodiments, anionic moieties may form a part of or be attached to the hydrophilic polymer or portion of a polymer. The anionic moieties or polymers containing the anionic moieties may be included in nanoparticles to tune the zeta potential, as desired. In embodiments, a hydrophilic polymer or portion of a polymer includes a hydroxyl group that can result in an oxygen anion when placed in a physiological aqueous environment. In embodiments, the polymer comprises PEG-OH where the OH serves as the charged moiety under physiological conditions.

Additional discussion of the role of zeta potential can be found in published PCT application, WO 2013/026299, entitled NANOPARTICLES FOR MITOCHONRIAL TRAFFICKING OF AGENTS, published on Aug. 22, 2013.

IX. Synthesis of Nanoparticle

Nanoparticles, as described herein, may be synthesized or assembled via any suitable process. Preferably, the nanoparticles are assembled in a single step to minimize process variation. A single step process may include nanoprecipitation and self-assembly.

In general, the nanoparticles may be synthesized or assembled by dissolving or suspending hydrophobic components in an organic solvent, preferably a solvent that is miscible in an aqueous solvent used for precipitation. In embodiments, acetonitrile is used as the organic solvent, but any suitable solvent may be used. Hydrophilic components are dissolved in a suitable aqueous solvent, such as water, 4 wt-% ethanol, or the like. The organic phase solution may be added drop wise to the aqueous phase solution to nanoprecipitate the hydrophobic components and allow self-assembly of the nanoparticle in the aqueous solvent.

A process for determining appropriate conditions for forming the nanoparticles may be as follows. Briefly, functionalized polymers and phospholipids may be co-dissolved in organic solvent mixtures (in embodiments, the phospholipids or functionalized phospholipids are dissolved in the aqueous solvent). This solution may be added drop wise into hot (e.g, 65° C.) aqueous solvent (e.g, water, 4 wt-% ethanol, etc.), whereupon the solvents will evaporate, producing nanoparticles with a hydrophobic core coated with phospholipids. The phospholipids used at this stage may be a mixture of non-functionalized phospholipids and functionalized phospholipids (e.g., conjugated to targeting moieties) than may also include a hydrophilic polymer component, such as PEG. Once a set of conditions where a high (e.g., >75%) level of targeting moiety surface loading has been achieved, contrast agents or therapeutic agents may be included in the nanoprecipitation and self-assembly of the nanoparticles.

If results are not desirably reproducible by manual mixing, microfluidic channels may be used.

NP properties may be controlled by (a) controlling the composition of the polymer solution, and (b) controlling mixing conditions such as mixing time, temperature, and ratio of water to organic solvent. The likelihood of variation in NP properties increases with the number of processing steps required for synthesis.

The size of the nanoparticle produced can be varied by altering the ratio of hydrophobic core components to amphiphilic shell components. The choice of PEGylated lipids and bilayer forming phoshpholipds can affect resulting nanoparticle size. PEGylated lipids are known to form small micellar structures because of surface tension imposed by the PEG chains. NP size can also be controlled by changing the polymer length, by changing the mixing time, and by adjusting the ratio of organic to the phase. Prior experience with NPs from PLGA-b-PEG of different lengths suggests that NP size will increase from a minimum of about 20 nm for short polymers (e.g. PLGA₃₀₀₀-PEG₇₅₀) to a maximum of about 150 nm for long polymers (e.g. PLGA_{100,000}-PEG_{10,000}). Thus, molecular weight of the polymer will serve to adjust the size.

NP surface charge can be controlled by mixing polymers with appropriately charged end groups. Additionally, the composition and surface chemistry can be controlled by mixing polymers with different hydrophilic polymer lengths, branched hydrophilic polymers, or by adding hydrophobic polymers.

Once formed, the nanoparticles may be collected and washed via centrifugation, centrifugal ultrafiltration, or the like. If aggregation occurs, NPs can be purified by dialysis, can be purified by longer centrifugation at slower speeds, can be purified with the use surfactant, or the like.

Once collected, any remaining solvent may be removed and the particles may be dried, which should aid in minimizing any premature breakdown or release of components. The NPs may be freeze dried with the use of bulking agents such as mannitol, or otherwise prepared for storage prior to use.

Useful discussion of processes for synthesizing nanoparticles is presented in, for example, published PCT patent application, WO 2013/033513, APOPTOSIS-TARGETING NANOPARTICLES, 7 Mar. 2013 and published PCT application, WO 2013/026299, entitled NANOPARTICLES FOR MITOCHONRIAL TRAFFICKING OF AGENTS, published on Aug. 22, 2013.

X. Use

In general, a nanoparticle as described herein may be used for any suitable purpose. The nanoparticles may be used for visualization, imaging, monitoring, diagnosis, or treating. In some embodiments, a nanoparticle described herein is used to treat a condition associated with dysfunction in lipid homeostasis or is used to maintain lipid homeostasis. In some embodiments, a nanoparticle as described herein is used to treat atherosclerotic plaques.

For purposes of treating or preventing a disease, an effective amount of a nanoparticle may be administered to a subject in need thereof. An “effective amount” is the quantity of nanoparticle in which a beneficial clinical outcome is achieved when the nanoparticle is administered to a subject. The precise amount of a nanoparticle administered to a subject will depend on the type and severity of the disease or condition and on the characteristics of the subject, such as general health, age, sex, body weight and tolerance to drugs. It may also depend on the degree, severity and type of disease. The skilled artisan will be able to determine appropriate dosages depending on these and other factors. Effective amounts of the disclosed nanoparticles may range between about 1 mg/mm² per day and about 10 grams/mm² per day Of course, effective amounts may fall outside of this range.

In various embodiments, a nanoparticle as described herein can be included in a pharmaceutical composition that can also include a pharmaceutically acceptable carrier or diluent. Suitable pharmaceutically acceptable carriers may contain inert ingredients that preferably do not inhibit the biological activity of a nanoparticle. Pharmaceutically acceptable carriers are preferably biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, Mack Publishing Company, Easton, Pa. Formulation of the compound to be administered will vary according to the route of administration selected (e.g., solution, emulsion, capsule). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrins) are known in the art (Baker, et al., “Controlled Release of Biological Active Agents”, John Wiley and Sons, 1986).

A nanoparticle can be administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds of the invention can also be administered orally (e.g., dietary), topically, by inhalation (e.g., intrabronchial, intranasal, oral inhalation or intranasal drops), or rectally, depending on the type of cancer to be treated.

### Definitions

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “treat” or the like means to cure, prevent, or ameliorate one or more symptom of a disease or condition.

As used herein, “lipid” includes phospholipid.

“Peptide” and “polypeptide” are used interchangeable herein.

As used herein, a compound that is “hydrophobic” is a compound that is insoluble in water or has solubility in water below 1 microgram/liter.

As used herein a compound that is “hydrophilic” is a compound that is water soluble or has solubility in water above 10 mg/liter.

As used herein, “bind,” “bound,” or the like means that chemical entities are joined by any suitable type of bond, such as a covalent bond, an ionic bond, a hydrogen bond, van der walls forces, or the like. “Bind,” “bound,” and the like are used interchangeable herein with “attach,” “attached,” and the like.

As used herein, a molecule to moiety “attached” to a core of a nanoparticle may be embedded in the core, contained within the core, attached to a molecule that forms at least a portion of the core, attached to a molecule attached to the core, or directly attached to the core. Accordingly, a molecule or moiety such as an HDL component that is attached to the core can be present at the core, at an outer surface of the nanoparticle or between the core and the outer surface of the nanoparticle.

As used herein, a “derivative” of a compound is a compound structurally similar to the compound of which it is a derivative. Many derivatives are functional derivatives. That is, the derivatives generally a desired function similar to the compound to which it is a derivative. By way of example, mannose is described herein as a macrophage targeting moiety because mannose binds macrophage mannose receptors. Accordingly, a functional mannose derivative is a mannose derivative that may bind a macrophage mannose receptor with the same or similar affinity as mannose (e.g., has dissociation constant that is within about a 100 fold range of that of mannose, such as within about a 10 fold range of that of mannose). By way of further example, triphenylphosophonium (TPP) cation is described herein as a mitochondrial targeting moiety because it can accumulate, or cause a compound or complex (such as a nanoparticle) to which it is bound to accumulate, in the mitochondrial matrix. Accordingly, a functional derivative of TPP is a derivative of TPP that may accumulate, or cause a compound or complex to which it is bound to accumulate, in the mitochondrial matrix in a similar concentration as TPP (e.g., within about a 100 fold concentration range, such as within about a 10 fold concentration range).

In the following, non-limiting examples are presented, which describe various embodiments of representative nanoparticles, methods for producing the nanoparticles, and methods for using the nanoparticles.

### Examples

Results and Discussion

Mitochondria Targeted HDL Mimicking NP for Efficient Cholesterol Efflux.

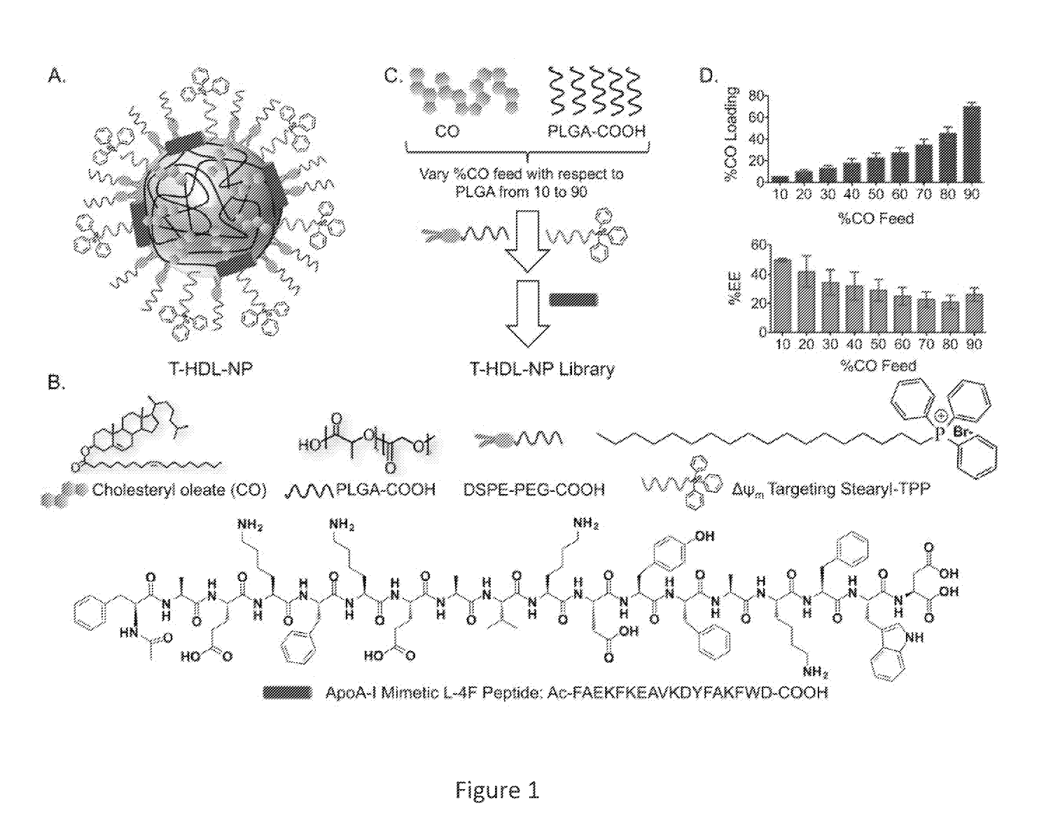

As described in Marrache, S.; Dhar, S. Biodegradable Synthetic High-Density Lipoprotein Nanoparticles for Atherosclerosis. Proc Natl Acad Sci USA 2013, 110, 9445-9450, a mitochondria-targeted HDL-mimicking polymer-lipid hybrid NP (T-HDL-NP) with a hydrophobic core containing cholesteryl oleate (CO) and biodegradable and FDA approved poly(lactic-co-glycolic acid) (PLGA) polymer was recently developed (FIG. 1A). This core was surrounded by a 1,2-distearoyl-sn-glycero-3-phosphoethanolamine (DSPE)-PEG lipid layer embedded with CO, apoA-I mimetic L-4F peptide with a Ac-FAEKFKEAVKDYFAKFWD (SEQ ID NO:1) sequence, stearyl-triphenyl phosphonium (TPP) cation with lipophilic, delocalized TPP cation for mitochondria targeting by taking advantage of the substantial negative mitochondrial membrane potential (Δψm) that exists across the IMM (FIG. 1B). For non-targeted control

(NT-HDL-NP), polyvinyl alcohol (PVA) was used instead of stearyl-TPP. These NPs were in the size range of 100-150 nm, which is ˜10 times larger than that of natural HDL which was advantageous to avoid rapid clearance by the kidneys or through extravasation and these NPs demonstrated long circulation with favorable biodistribution (bioD) and pharmacokinetic (pK) parameters. Analyses of the NPs demonstrated composition similarities with natural HDL and T-HDL-NPs were found to be non-toxic and nonimmunogenic. T-HDL-NPs demonstrated unique abilities to target mitochondria of macrophages and within the mitochondria, T-HDL-NPs were localized mainly in the mitochondrial matrix and the intermembrane space (IMS) indicating that this T-HDL-NP has the potential to be able to participate in intracellular cholesterol transport pathway.

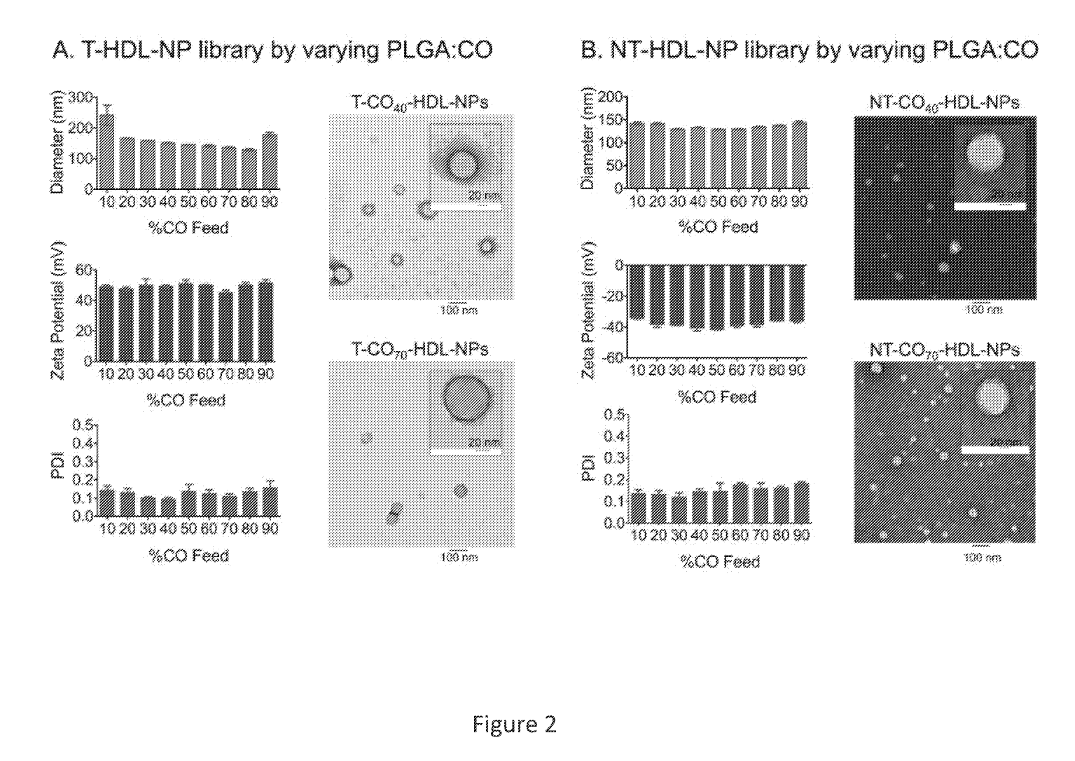

Preliminary studies with 22-(N-(7-nitrobenz-2-oxa-1,3-diazol-4-yl)amino)-23,24-bisnor-5-cholen-3β-ol (NBD-cholesterol) indicated cholesterol binding properties of mitochondria-targeted (T)-HDL-NPs. We synthesized a library of T-HDL-NPs by varying the ratio of cholesterol oleate (CO) to PLGA to determine effective CO and apoAI loading ratios with suitable size and zeta potential for maximum cholesterol binding (FIG. 1C). As the percent feed of CO was increased, % CO and % apoA-I loadings were increased, % CO encapsulation efficiency (EE) decreased with increased CO feed (FIG. 1D, Table S1). However, the size and zeta potential trends of this NP library indicated that by adding suitable amount of PLGA, we are able to achieve stable NPs with suitable size and charge (FIG. 2A, FIG. S1, Table S1). A library of non-targeted (NT)-HDL-NPs was constructed from PLGA-COOH, CO, PVA, DSPE-PEG-COOH, and 4F peptide through a modified nanoprecipitation as described for T-HDL-NPs by varying the ratio of PLGA to CO (Table S2, FIG. 2B, Figure S2). NT-HDL-NPs demonstrated similar size ranges as seen with T-HDL-NPs; the NT-HDL-NPs had negative zeta potential. Morphology of the library of T-HDL-NPs was confirmed by transmission electron microscopy (TEM) (FIG. 2A, FIG. S3). TEM studies with this library indicated similar morphology across the NT-HDL-NPs. A comparison of morphology of T and NT-HDL NPs indicated similar pattern (FIGS. 1, 2, FIGS. S1, S3 and S4).

HDL Mimicking NP for Cholesterol Binding and Efflux.

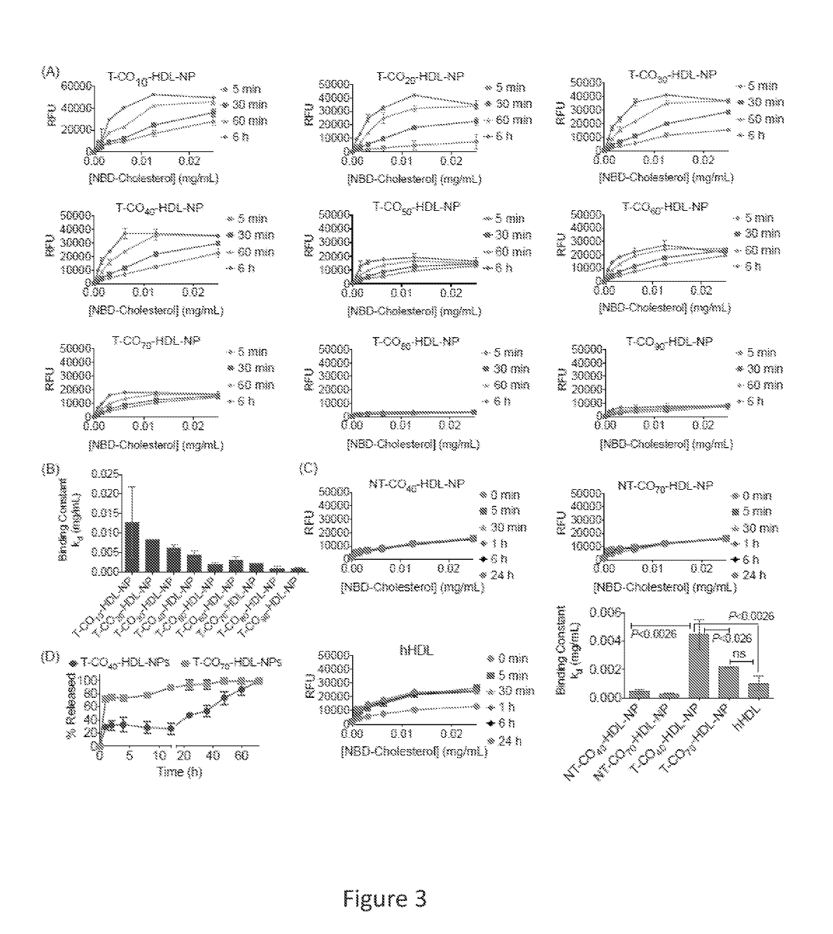

In vitro cholesterol binding assay using NBD-cholesterol with the NP library provided insight pertaining to CO:PLGA ratio to maximize the efficiency of cholesterol binding ability of T-HDL-NPs (FIG. 3A). A time dependent cholesterol binding assay indicated that the T-HDL-NPs with low % CO feed with respect to PLGA demonstrated the highest cholesterol binding ability. As the % CO feed was increased, cholesterol-binding property was reduced (FIG. 3A).

Binding constant (kd) determination by analyzing the binding curves obtained from T-HDL-NP library at a constant time point of 6 h with “one site total binding” function in GraphPad Prism 5.0 software using the equation: RFU=(Bmax*[NBD−cholesterol])/(kd+[NBD−cholesterol]) indicated that cholesterol binding is high at low CO feed and the binding pattern is moderate in the range of 40-70% CO feed (FIG. 3B). NBD-cholesterol binding abilities of human HDL (hHDL) was also studied to serve as a control (FIG. 3C). The NT-HDL-NPs demonstrated significantly reduced cholesterol binding properties compared to T-HDL-NPs (FIG. 3C, FIG. S5, FIG. S6). This further supported that the use of mitochondria targeted HDL mimicking NP can be beneficial in cholesterol efflux. A comparison of kd values of T-CO40-HDL-NPs, T-CO70-HDL-NPs, NT-CO40-HDL-NPs, NT-CO70-HDL-NPs, and hHDL demonstrated remarkable cholesterol binding abilities of T-HDL-NPs comparable to hHDL and significantly higher than the corresponding NT-HDL-NPs (FIG. 3D). We also would like to stress that gold-core based HDL NPs have been reported in the literature to have binding constants in the lower nanomolar range. NPs with 40-70% CO feed showed favorable NP size and therefore taking the size and cholesterol binding together, we decided to use NPs with 40% (T-CO40-HDL-NP) and 70% (T-CO70-HDL-NP) CO feed for our future studies. The mitochondria targeted T-HDL-NPs with showed binding constants in the high micromolar range and this property might translate to in vivo efficacious RCT properties.

We investigated the release kinetics of bound NBD-cholesterol from the T-HDL-NPs (FIG. 3D). Release of bound NBD-cholesterol from T-HDL-NPs indicated that T-CO40-HDL-NPs with less CO content release bound cholesterol in a more controlled fashion compared to T-CO70-HDLNPs which had 70% CO feed under physiological pH of 7.4 at 37° C. (FIG. 3D). Since these two formulations showed differences in cholesterol binding abilities and differential release kinetics of bound cholesterol and hence these two formulations can be unique in showing unique therapeutic potential when used in vivo. Therefore, we decided to carry out in vitro and in vivo therapeutic studies with these two formulations.

Therapeutic Potential of T-HDL-NPs Using Foam Cells.

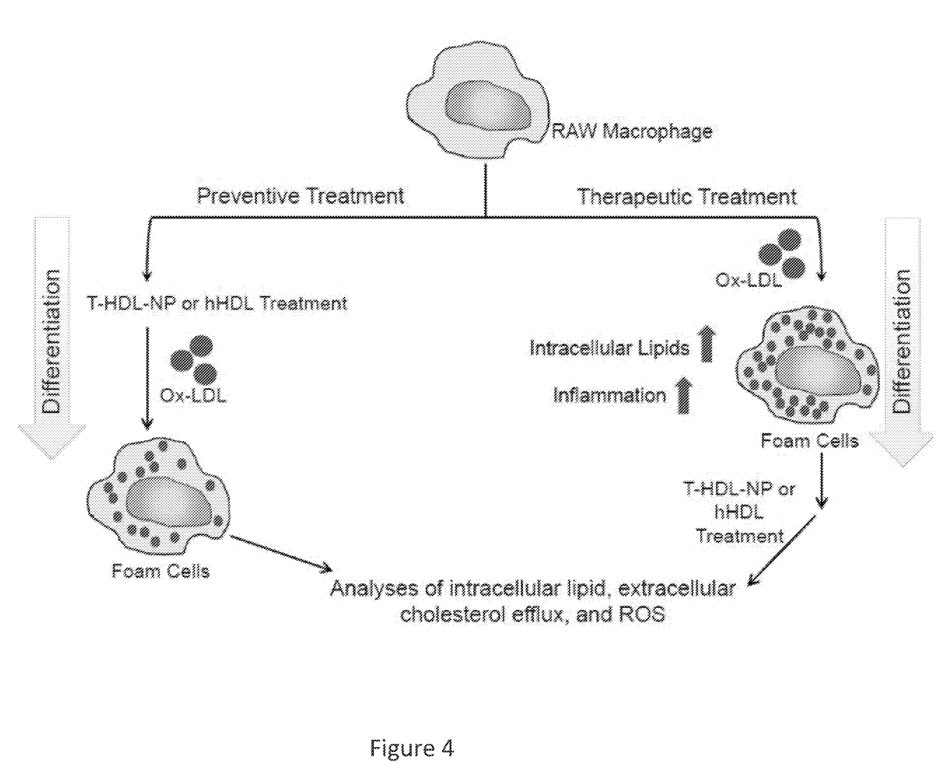

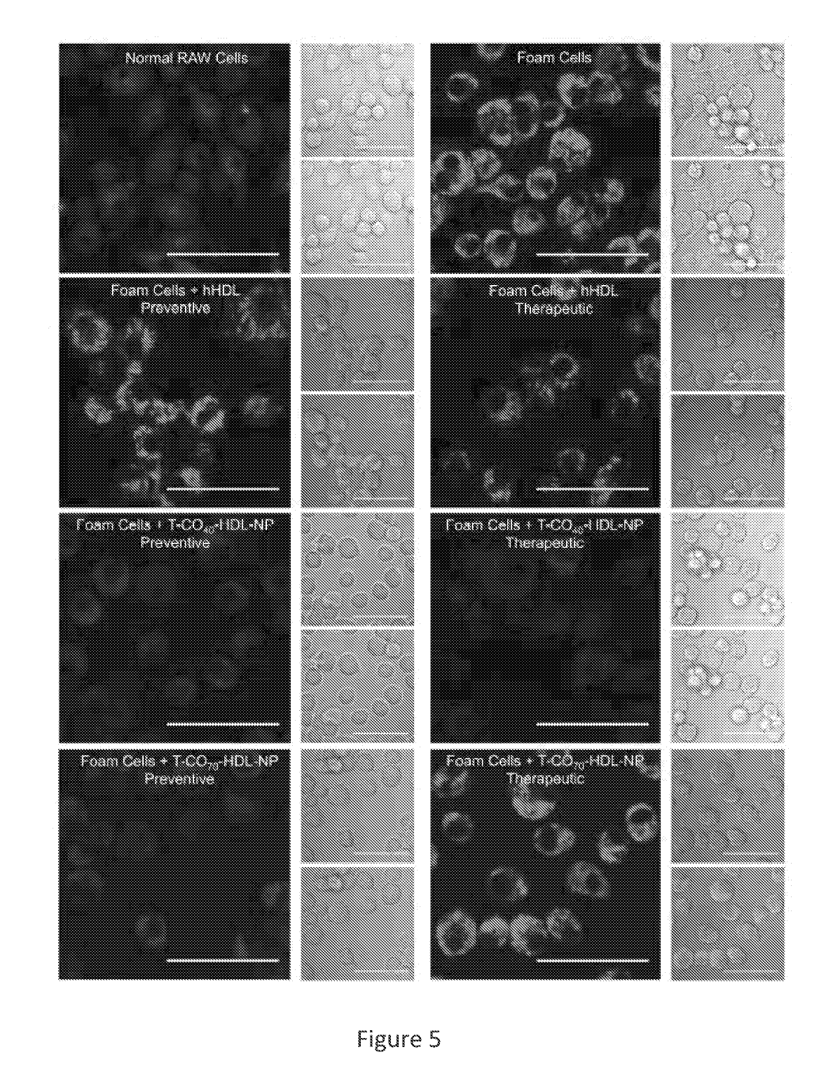

Cholesterol accumulation in arterial wall macrophages leads to foam cell formation. This step is one of the initial steps of atherogenesis. During foam cell formation, cholesterol influx and efflux balance in macrophages becomes dysfunctional. Under well-maintained cholesterol homeostasis environment, the balance between cholesterol influx and efflux depends on limiting cholesterol inflow through scavenger receptors and outflow through RCT to transport the excess cholesterol from laden macrophages in vessel walls to the liver for excretion. A number of proteins such as cytochrome P450 cholesterol 27-hydroxylase, ABC transporters, and liver X receptors work together to accomplish RCT. A step in RCT is the transfer of cholesterol to lipid-poor molecules in the plasma such as apoA-I containing HDL. Macrophages rely on RCT to remove excess cholesterol. Reduction of cholesterol accumulation in the artery wall can slow or prevent development of atherosclerosis. We investigated atheroprotective effects of T-HDL-NPs on RAW 264.7 macrophage-derived foam cells formed by oxidized LDL (oxLDL). RAW macrophages were treated with oxLDL (100 μg/mL) to induce foam cell formation (FIG. 4). Foam cells derived from macrophages in presence of oxLDL demonstrate inflammatory signaling, cholesterol accumulation, and oxidative stress similar to the hallmark of early atherosclerotic lesions. T-CO40-HDL-NPs and T-CO70-HDL-NPs were used and NP treatment was carried out 6 h prior and after differentiation to gain insight about both preventive and therapeutic potential of these NPs. As controls, hHDL isolated from blood bank produced human plasma was used under both therapeutic and preventive settings. Effects on total cholesterol, triglyceride, anti-inflammatory, and anti-oxidative effects of these NPs and hHDL on foam cells were studied (FIG. 4).

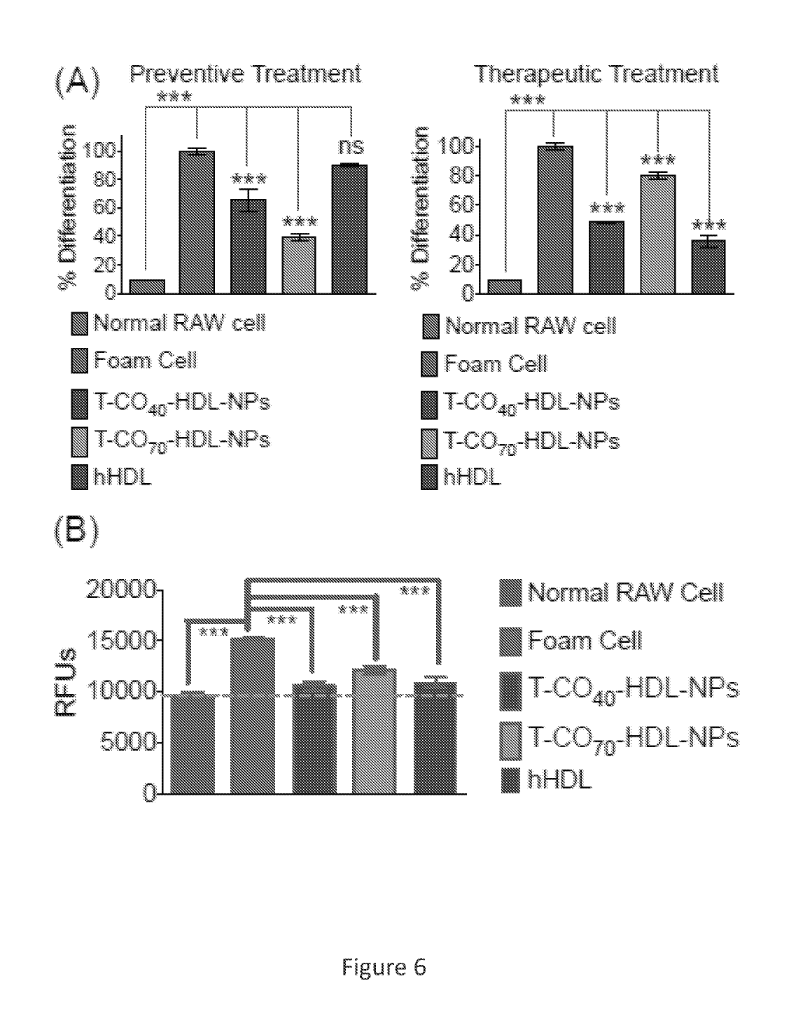

Intracellular triglyceride was stained using AdipoRed and live cell imaging was performed to analyze the lipid droplets in foam cells in the absence and presence of T-HDLNPs or hHDL (FIG. 5). Both methods of treatment indicated that T-HDL-NP have the ability to reduce lipid levels in macrophages as seen by live cell imaging of lipid droplets in these cells (FIG. 5). Both methods of treatment indicated that T-CO40-HDL-NPs have the ability to reduce lipid levels in macrophages. Lipid reduction abilities of T-CO70-HDL-NP were found to be better under preventive settings compared to abilities seen under therapeutic treatment (FIG. 5). Native hHDL demonstrated more efficient lipid reduction profiles under therapeutic treatment (FIG. 5). Quantification of intracellular triglyceride contents using AdipoRed assay was used to quantify the extent of differentiation of macrophages into foam cells in presence of oxLDL (FIG. 6A).

Both methods of treatment indicated that T-CO40-HDL-NPs and T-CO70-HDL-NPs have the ability to reduce differentiation of macrophages into foam cells (FIG. 6A). Native hHDL was able to stop this differentiation only under therapeutic settings (FIG. 6A). Quantification of the reactive oxygen species (ROS) levels in the foam cells demonstrated an elevated ROS levels in cells compared to normal macrophages (FIG. 6B). Treatment of foam cells with T-CO40-HDL-NPs or T-CO70-HDL-NPs or hHDL attenuated the increased ROS levels in foam cells. Reduction of ROS levels by the NPs was comparable to the effects shown by natural hHDL (FIG. 6B).

BioD and PK of T-HDL-NPs in Piglet.

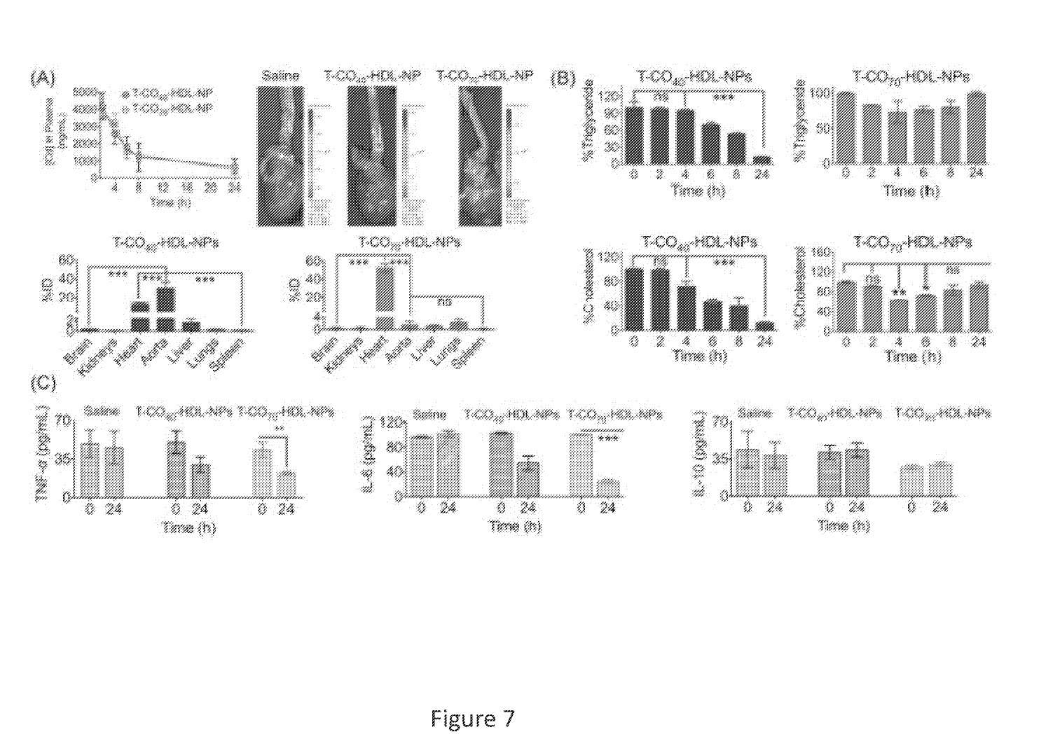

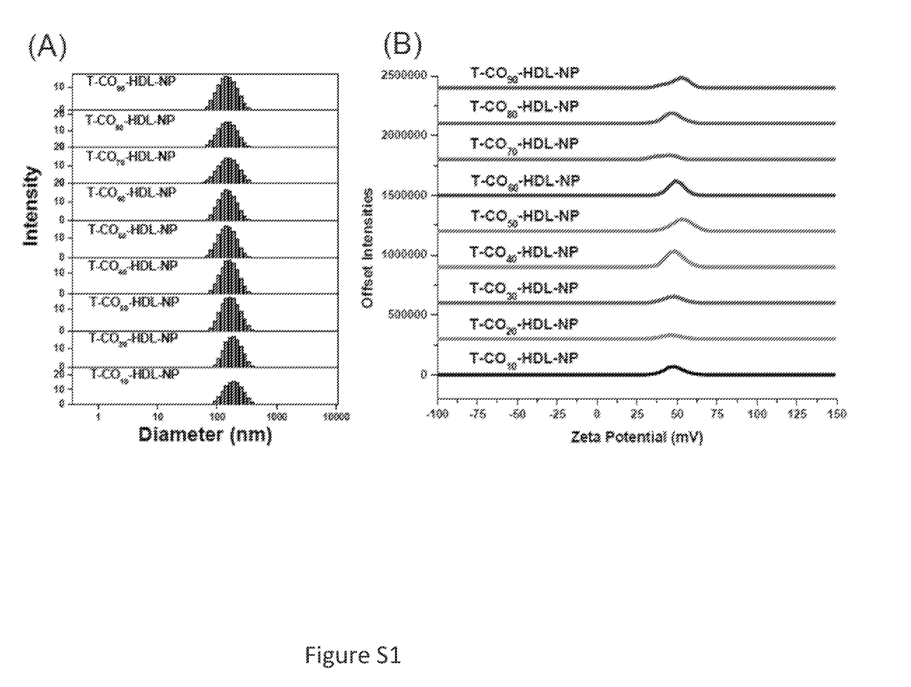

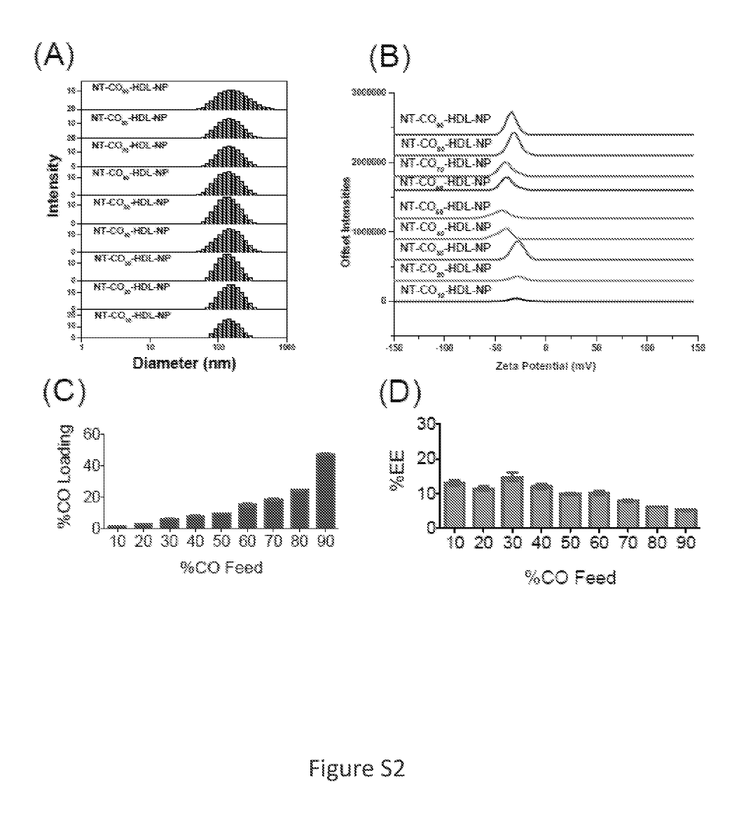

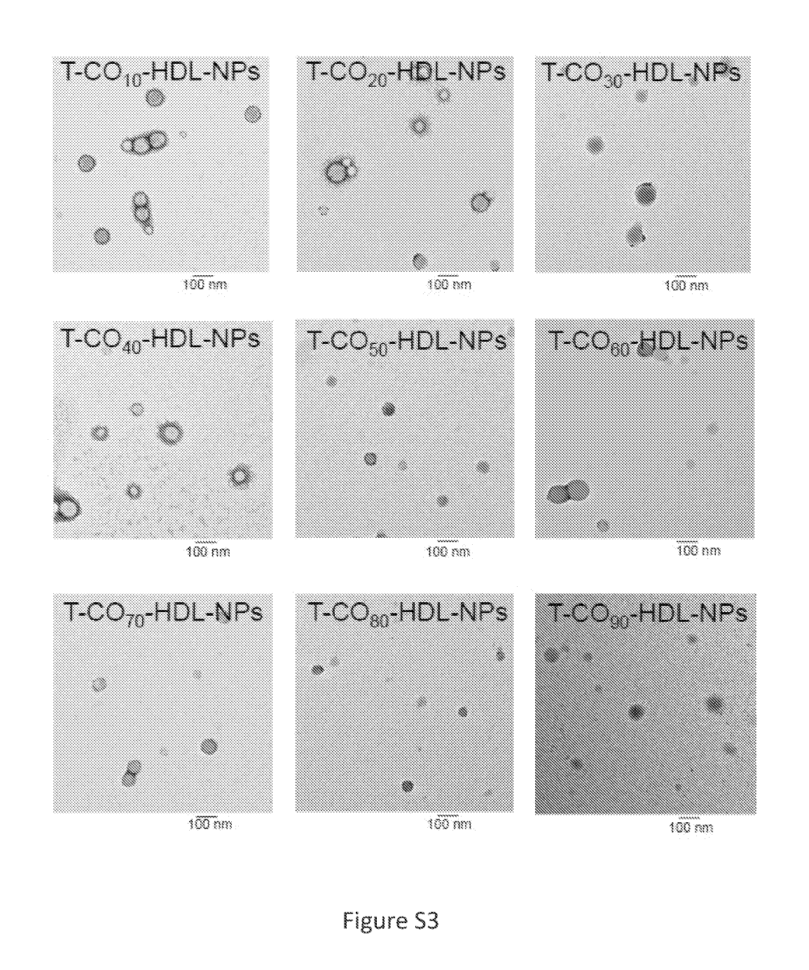

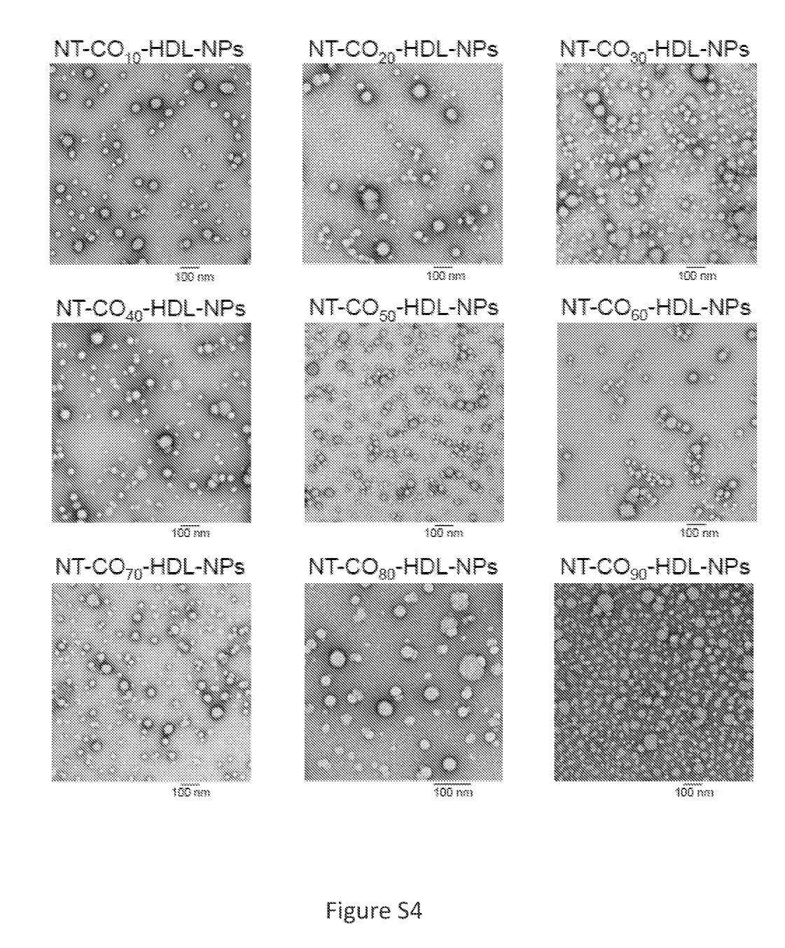

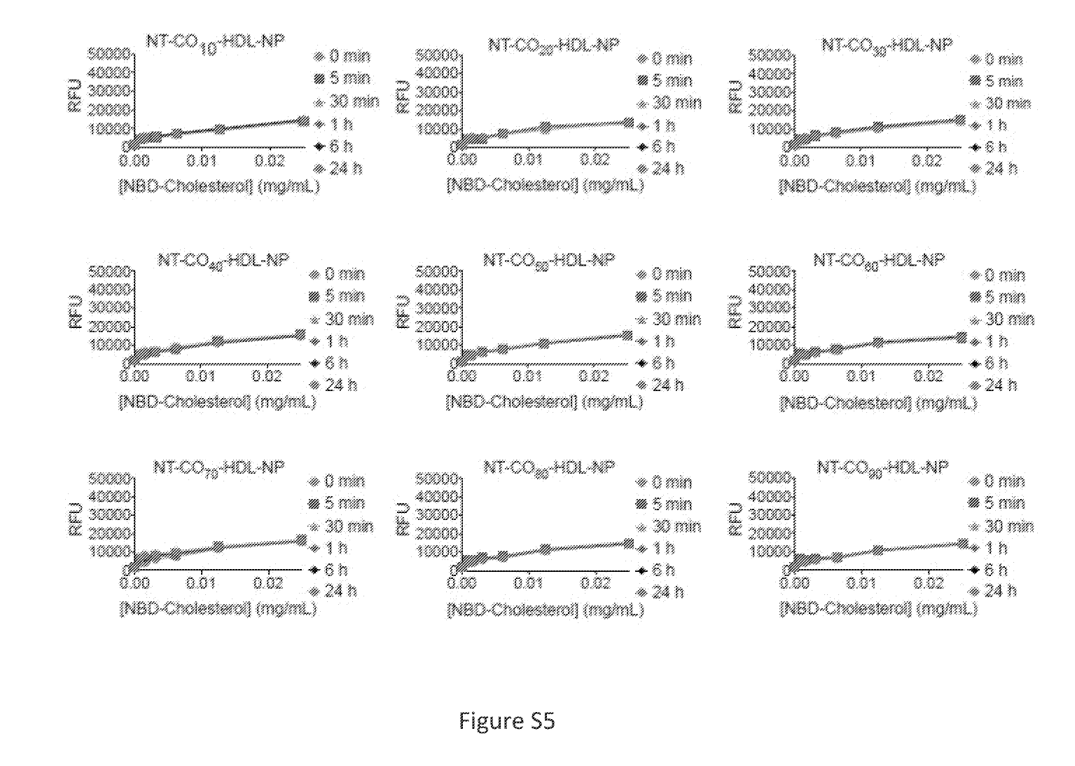

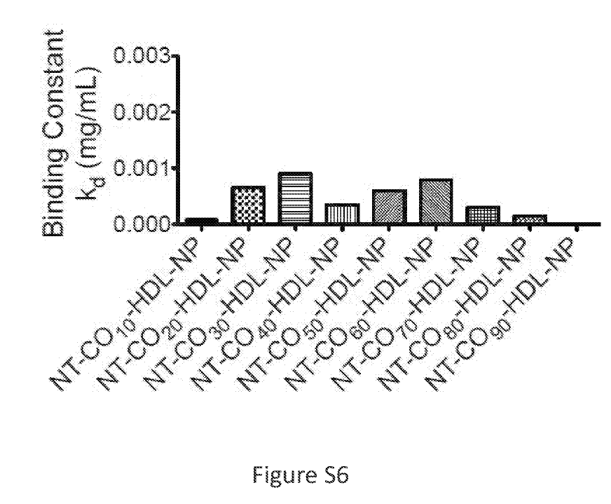

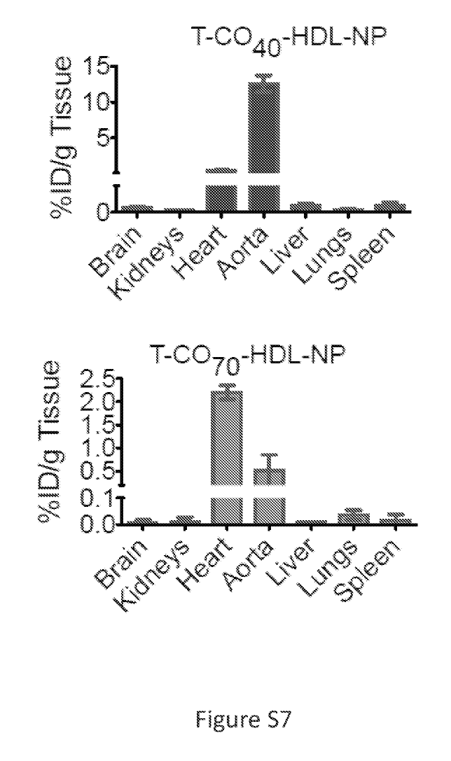

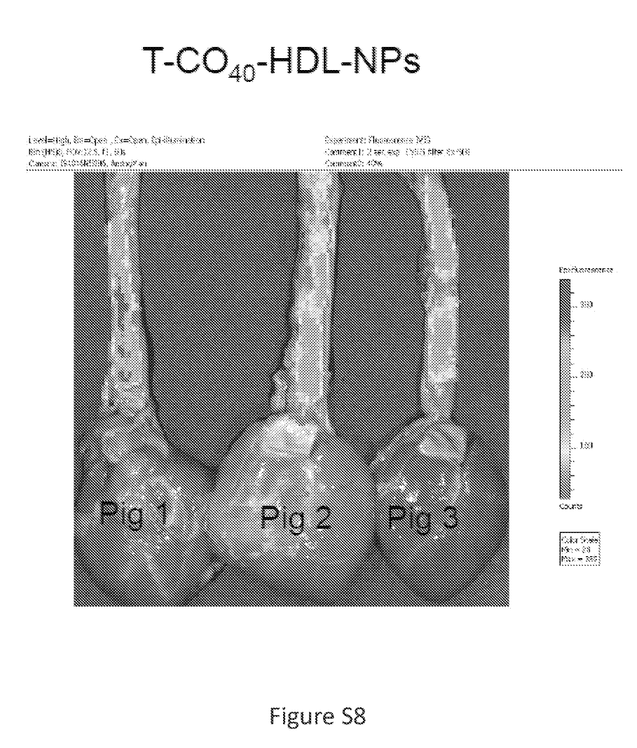

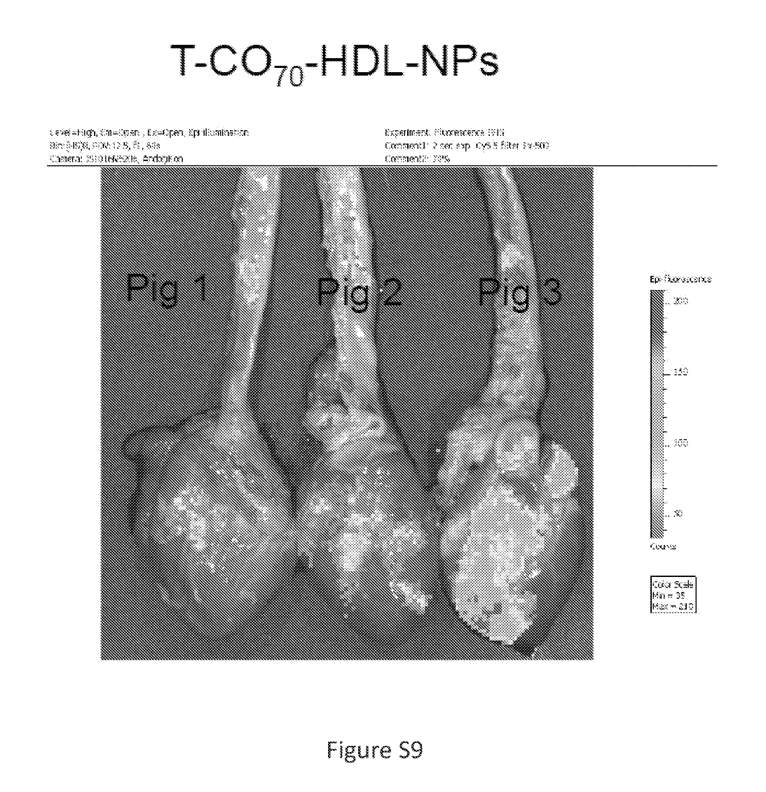

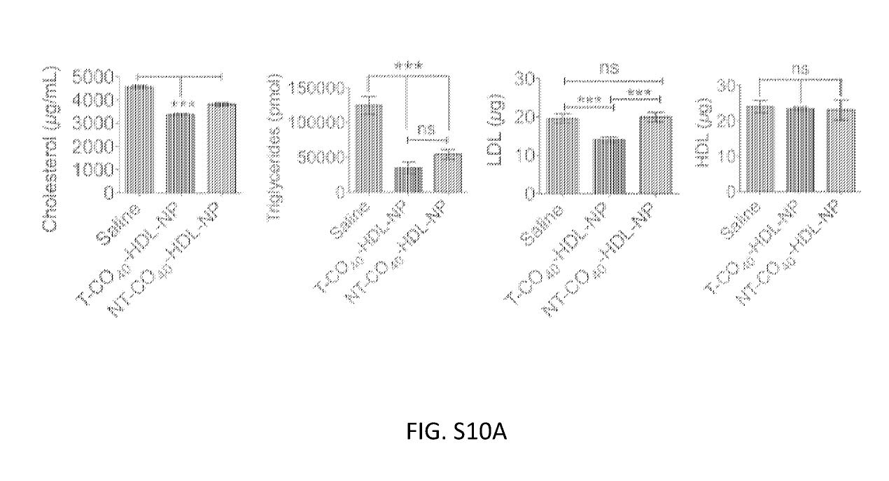

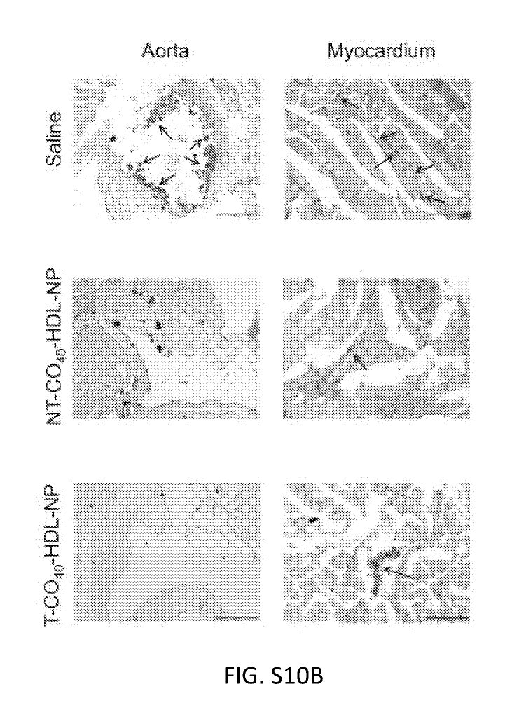

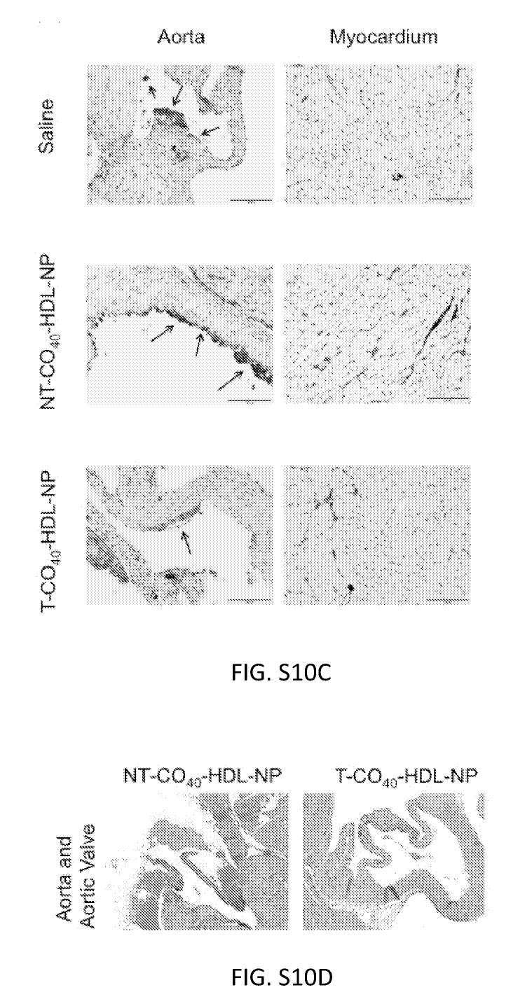

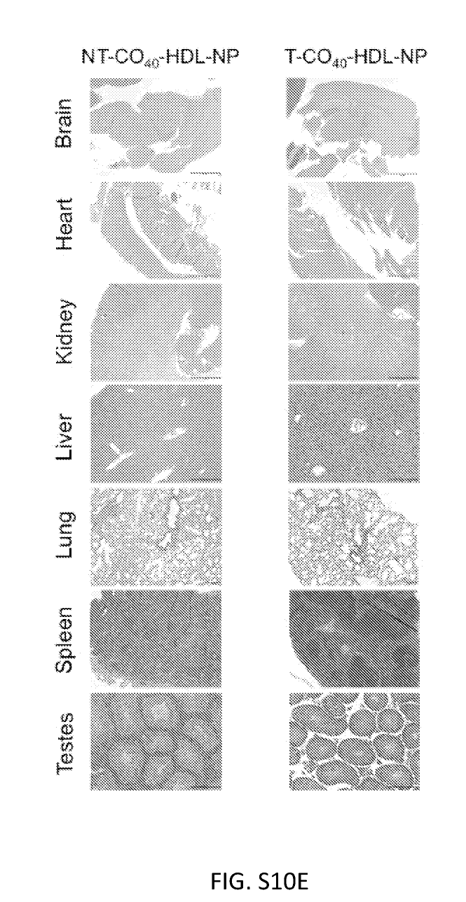

We (Marrache, S.; Dhar, S. Biodegradable Synthetic High-Density Lipoprotein Nanoparticles for Atherosclerosis. Proc Natl Acad Sci USA 2013, 110, 9445-9450) previously reported PK and bioD studies of T-CO75-HDL-NPs in Sprague Dawley rats, which demonstrated favorable PK and bioD profiles. The THDL-NPs were mostly distributed in the heart. However, the swine heart is more similar to humans. The distribution of blood supply by the coronary artery system in pig are almost identical to that of humans. The size of the heart and blood vessels in pigs are more analogous to humans than either the dog, rabbit or nonhuman primate. These are all factors that will likely effect therapeutic outcomes and suggest that results found in the pig maybe more translatable to humans. We therefore, assessed PK and bioD properties of two carefully selected formulations T-CO40-HDL-NP and T-CO70-HDL-NPs in piglets. T-CO40-HDL-NP or T-CO70-HDL-NPs containing quantum dots (QDs) for detection were administered in American Landrace piglets (4 weeks old) at a dose of 1.25 mg/kg with respect to total NP (0.181 mg/kg for 40%-CO and 0.165 mg/kg for 70%-CO with respect to Cd) by intravenous injection (FIG. 7A). We used a polymer conjugated QD, PLGA-PEG-QD in these NP formulations. Analysis of the plasma samples for Cd by ICP-MS and quantification of PK parameters by a one-compartment intravenous input model revealed very similar PK profiles for both the formulations (FIG. 7A, Table 1). Both the formulations demonstrated favorable PK parameters as evidenced by long elimination half-life (t½), lower clearance (CL), and very high area under curve (AUC). In addition, maximum serum concentration (Cmax) indicated that these NPs are delivered into the bloodstream very effectively even in a larger animal model such as piglets. Animals were sacrificed 24 h post administration and bioD was studied by performing fluorescence microscopy using in vivo imaging system (IVIS) and by quantifying Cd in the different organ samples by inductively coupled plasma mass spectrometry (ICP-MS) (FIG. 7A). Fluorescence microscopy using IVIS demonstrated that the T-CO40-HDLNPs were mostly distributed in the aorta and heart, T-CO70-HDL-NPs were mostly distributed in the heart (FIG. 7A). The distribution profiles of T-CO40-HDL-NP and T-CO70-HDL-NP in tissues such as brain, kidneys, heart, aorta, liver, lungs, and spleen at 24 h post-dose varied significantly by ICP-MS further supported differential distribution of the two NP formulations (FIG. 7A). Quantitative analyses showed that T-CO40-HDL-NP were mostly distributed in the aorta and heart, 30±6.5% of the injected dose (ID) was found in the aorta and 13.3±1.15% of ID was found in the heart (FIG. 7A). The T-CO70-HDL-NP were mostly distributed in the heart, 51.6±4.7% of ID was found in the heart and only 1.48±1.17% of ID was found aorta. We also observed differences in the % ID per gram of tissue (% ID/g) in brain, kidneys, heart, aorta, liver, lungs, and spleen (FIG. S7). This differential distribution of T-CO40-HDL-NP and T-CO70-HDL-NP was consistent across all animals studied (FIGS. S8 and S9). The differential distribution of the two formulations indicated that these NPs might show different therapeutic and lipid reduction profiles.

Lipid Reduction by T-HDL-NPs in Piglet. Total cholesterol and triglyceride levels of the piglets treated with T-CO40-HDL-NP and T-CO70-HDL-NP showed very different profiles (FIG. 7B). Time dependent lipid profiles were analyzed after administration of both the NPs. The T-CO70-HDL-NPs resulted in a temporary reduction of cholesterol at 4 h, levels returned to a pretreatment state after 8 h. The total cholesterol in the piglets treated with T-CO40-HDL-NP was reduced dramatically after 24 h. A similar pattern was observed in the triglyceride levels in these animals (FIG. 7B). We believe that greater retention of T-CO40-HDL-NP in the aorta, the plaque accumulation location and ability to uptake cholesterol more efficiently might be responsible for its remarkable efficiency in reduction of cholesterol and triglyceride levels.

Anti-inflammatory Properties of T-HDL-NPs in Piglet. Natural HDL is known to show anti-inflammatory characteristics. Pro-inflammatory interleukin (IL)-6 and tumor necrosis factor (TNF)-α cytokine levels and anti-inflammatory IL-10 levels were determined in the plasma samples from the pigs treated with T-CO40-HDL-NPs and T-CO70-HDL-NPs (FIG. 7C). These two formulations demonstrated different anti-inflammatory properties. Reduction of pro-inflammatory cytokines by T-CO40-HDL-NPs was less compared to T-CO70-HDL-NPs and no significant changes were observed in the IL-10 levels with any of these formulations. Since these studies were carried out in normal pigs only for 24 h, there are likely only baseline cytokine levels during the study period and hence these NPs did not show changes in IL-10 levels.

TABLE 1
PK Profile of T-HDL-NPs in Pig

| | | | One Compartment Model^{[1]} | | |
|---|---|---|---|---|---|
| | AUC_{[0-24 h]} | Cmax, | V_{d} | C_{L} t = 0 | |
| Group | (ng ML⁻¹h) | ng mL−1 | (mL · kg⁻¹) | (mL/Kg h) | T_{1/2} (h) |
| T-CO40- | 31,973 ± 12,186 | 6,244 ± 1,405 | 29.9 ± 6.1 | 6.1 ± 2.3 | 3.9 ± 2.3 |
| HDL-NP | | | | | |
| T-CO70- | 31,544 ± 4,742 | 5,411 ± 668 | 30.9 ± 3.7 | 5.2 ± 0.8 | 4.2 ± 0.9 |
| HDL-NP | | | | | |

^{[1]}Least-squares fit to model: C = A*exp[−k1*t)

Therapeutic Efficacy in apoE^{-/-} Mice.

Therapeutic efficacy of T-CO₄₀-HDL-NP and NT-CO₄₀-HDL-NP were tested in apoE^{-/-} mouse model fed with normal diet. Animals were treated T-CO₄₀-HDL-NPs or NT-CO₄₀-HDL-NPs at a dose of 10 mg/kg (with respect to total NP) twice weekly via intravenous injection. Plasma lipid levels were compared between the treatments by quantifying triglyceride, total cholesterol, LDL, and HDL. Results are shown in FIG. S10A.

Oil red O staining of aortic valves from saline, T-CO₄₀-HDL-NP, and NT-CO₄₀-HDL-NP treated apoE^{-/-} animals are shown in FIG. S10B. The amount of cleaved caspase-3-positive areas from aorta and myocardium from saline, T-CO₄₀-HDL-NP, and NT-CO₄₀-HDL-NP treated apoE^{-/-} animals are shown in FIG. S10C. H and E stained aorta and aortic valve from T-CO₄₀-HDL-NP and NT-CO₄₀-HDL-NP treated apoE^{-/-} animals are shown in FIG. S10F. H and E stained images of all major organs from T-CO₄₀-HDL-NP and NT-CO₄₀-HDL-NP treated apoE^{-/-} animals are shown in Figure S10E, demonstrating no significant toxicity.

The table below show the characterization of the nanoparticles used in the study testing the therapeutic efficacy in apoE^{-/-} mice.

TABLE
Characterization of NPs used in ApoE^{-/-}-Normal Diet Study

| | | | | Zeta |
|---|---|---|---|---|
| Injection | Sample | Z-Ave | | Potential |
| Number | Name | (d · nm) | PDI | (mV) |
| 1 | T-CO₄₀- | 146.9 ± 0.7 | 0.112 ± 0.013 | 29.8 ± 1.9 |
| | HDL-NPs | | | |
| 1 | NT-CO₄₀- | 130.1 ± 3.3 | 0.162 ± 0.017 | −25.4 ± 1.3 |
| | HDL-NPs | | | |
| 2 | T-CO₄₀- | 178.7 ± 1.2 | 0.144 ± 0.017 | 20.6 ± 0.7 |
| | HDL-NPs | | | |
| 2 | NT-CO₄₀- | 160.60 ± 0.7 | 0.123 ± 0.006 | −33.0 ± 2.2 |
| | HDL-NPs | | | |
| 3 | T-CO₄₀- | 177.5 ± 2.8 | 0.119 ± 0.010 | 16.8 ± 1.8 |
| | HDL-NPs | | | |
| 3 | NT-CO₄₀- | 179.0 ± 14.1 | 0.141 ± 0.011 | −30.0 ± 0.7 |
| | HDL-NPs | | | |
| 4 | T-CO₄₀- | 175.4 ± 1.0 | 0.087 ± 0.007 | 30.2 ± 2.9 |
| | HDL-NPs | | | |
| 4 | NT-CO₄₀- | 177.7 ± 1.3 | 0.130 ± 0.007 | −34.9 ± 2.2 |
| | HDL-NPs | | | |
| 5 | T-CO₄₀- | 173.1 ± 2.3 | 0.110 ± 0.017 | 21.7 ± 1.2 |
| | HDL-NPs | | | |
| 5 | NT-CO₄₀- | 186.4 ± 2.9 | 0.175 ± 0.023 | −28.0 ± 0.2 |
| | HDL-NPs | | | |
| 6 | T-CO₄₀- | 158.1 ± 1.4 | 0.111 ± 0.011 | 27.1 ± 2.1 |
| | HDL-NPs | | | |
| 6 | NT-CO₄₀- | 189.4 ± 3.4 | 0.180 ± 0.007 | −27.1 ± 0.5 |
| | HDL-NPs | | | |
| 7 | T-CO₄₀- | 164.6 ± 1.4 | 0.093 ± 0.024 | 41.2 ± 1.7 |
| | HDL-NPs | | | |
| 7 | NT-CO₄₀- | 186.4 ± 3.0 | 0.175 ± 0.023 | −28.0 ± 0.2 |
| | HDL-NPs | | | |

Conclusions

The NP platform presented here could decrease plaque inflammation directly by locally accumulating HDL mimic in plaque macrophages, working at the intracellular cholesterol metabolism pathways by targeting the mitochondria. Delivery of cholesterol to the mitochondria is the rate-limiting step for cholesterol degradation in the liver. Therefore, mitochondria targeted THDL-NPs will be able to carry excess cholesterol from cytosol to the mitochondria to play an important role in the maintenance of both extra and intracellular lipid homeostasis. This technology uses biocompatible polymers and lipids to create a synthetic yet biodegradable HDL mimic. Cholesterol plays many well-described roles within the cell, but how cholesterol moves to and from key organelles to perform these roles is not as well known. The technology presented here is synthetic, biodegradable and the mitochondria targeted HDL mimicking NP has the potential in participating in intracellular lipid homeostasis.

### Experimental Section

Animals.

American Landrace piglets (4 weeks old) were obtained from the University of Georgia Swine Farm and handled in accordance with Animal Welfare Act (AWA), and other applicable federal and state guidelines. All animal work presented here was approved by Institutional Animal Care and Use Committee (IACUC) of UGA.

Synthesis of Library of T-HDL-NPs and NT-HDL-NPs with Varied CO Feed.

T-HDL-NPs were prepared via self-assembly of PLGA-COOH, CO, stearyl-TPP, DSPE-PEG-COOH, and apoA-I mimetic L-4F peptide through a modified nanoprecipitation. PLGA-COOH (10 to 90 μL, 25 mg/mL in CH₃CN) and CO (90 to 10 μL, 25 mg/mL in DMSO/THF=9:1) were mixed so that the total volume of PLGA and CO was 100 μL. Stearyl-TPP (5 mg/mL, 100 μL) and DSPE-PEG-COOH (1 mg/mL, 100 μL) with a weight ratio of 16% to the PLGA polymer were dissolved in 4% ethanol aqueous solution. To prepare the library of NT-HDL-NPs, PVA (5 mg/mL in 4% aqueous ethanol, 100 μL) was used instead of stearyl-TPP during the modified nanoprecipitation process. The lipid solution was heated to 65° C. to ensure all lipids are in the non-assembled state. The PLGA/CO solution was added into the preheated lipid solution drop-wise under vigorous stirring. The mixed solution was vortexed vigorously for 3 min followed by vigorous stirring for 2 h at room temperature. The remaining organic solvent and free molecules were removed by washing the NP solution three times using an Amicon Ultra-4 centrifugal filter with a molecular weight cutoff of 100,000 Da. The NPs were incubated with apoA-I mimetic L-4F peptide (1 mg/mL, 50 μL) at 4° C. for 12-14 h. The NPs were further washed three times using Amicon Ultra-4 centrifugal filter with a molecular weight cutoff of 100,000 Da to removed unbound peptide. NP size (diameter, nm), PDI, and surface charge (zeta potential, mV) were obtained from three independent measurements. For TEM studies, 8 μL of NP solution was diluted with 187 μL water, and then 5 μL of 4% uranyl acetate was added into the solution to stain the NPs. This mixture was vortexed for few sec and 4 μL of the mixture was dropped into a copper grid and the samples were dried. TEM images were recorded on FEI Tecnai transmission electron microscope operating at 200 kV. QD loading in the NPs was quantified by ICP-MS. The amount of cholesterol present in the NPs was determined by an Amplex Red assay.

Quantification of Cholesterol in NPs.

Cholesterol content of NPs was quantified by AmplexRed cholesterol quantification kit. NP solutions (50 μL) were incubated with a working reagent solution composed of AmplexRed (300 μL), horse radish peroxidase (HRP) (0.2 U/mL), cholesterol oxidase (2 U/mL), and cholesterol esterase (0.2 U/mL) for 30 min at 37° C. in the dark. The fluorescence was measured using a plate reader at an excitation of 560 nm and an emission of 590 nm. Relative fluorescence units (RFUs) were converted to cholesterol quantification using a standard curve of cholesterol oleate.

NBD-Cholesterol Binding Studies.

Cholesterol binding to T-HDL-NPs, NT-HDL-NPs, and hHDL was determined by adding 5 μL of varying concentrations of NBD-cholesterol (0, 0.00078, 0.00156, 0.00312, 0.00625, 0.0125, 0.025 mg/mL) in DMF to 995 μL of NPs (0.025 mg/mL) in water. The solutions were vortexed and incubated for 5 min, 30 min, 1 h, 6 h, and 24 h. The fluorescence was quantified by plate reader with an excitation wavelength 473 nm and emission of 560 nm.

Formation and Prevention of Foam Cell from RAW Macrophages.

RAW macrophages were plated on a 24 well plate at a density of 2.0×106/well in RPMI and allowed to grow to confluency. The media was removed and a lipid depleted DMEM (10% lipoprotein deficient FBS, 1% penicillin-streptomycin) media was added and the cells were grown for an additional 24 h. For preventative treatment, T-CO40-HDL-NPs or T-CO70-HDL-NPs or hHDL (0.1 mg/mL) were added and allowed to internalize for 6 h. The media was changed and fresh RPMI was added supplemented with oxidized LDL (Ox-LDL) (100 μg/mL). The cells were further incubated for 24 h. For therapeutic foam cell treatment, after 24 h treatment with lipid deprived media, the media was replaced with RPMI supplement with Ox-LDL (100 μg/mL) and the cells were incubated for 12 h. After which, the media was removed and T-CO40-HDL-NPs or T-CO70-HDL-NPs or hHDL (0.1 mg/mL) were added and allowed to internalize for 24 h. The media was removed for both cases, and washed with 1×PBS (3×). To image the foam cells, AdipoRed in PBS was added to each well and incubated for 10 min. The AdipoRed was removed and the cells were washed with 1×PBS (5×). The plates were then either read on the plate reader for the relative fluorescent units or image via confocal confocal microscopy (TRITC, 500 ms).

ROS Detection in Foam Cells.

RAW 264.7 macrophages were plated on a 24 well plate at a density of 2.0×106 cells/well in RPMI and allowed to grow to confluency. The media was removed and lipid depleted DMEM (10% lipoprotein deficient FBS, 1% penicillin-streptomycin) media was added and the cells were grown for an additional 24 h. The media was changed and fresh RPMI was added supplemented with Ox-LDL (100 μg/mL). The cells were further incubated for 24 h. After 24 h treatment with lipid deprived media, the media was replaced with RPMI supplement with Ox-LDL (100 μg/mL) and the cells were incubated for 12 h. After which, the media was removed and T-CO40-HDL-NPs or T-CO70-HDL-NPs or hHDL (0.1 mg/mL) were added and allowed to internalize for 24 h. After which, a dichlorodihydrofluoroscein diacetate (DCFH-DA) solution in RPMI was added and incubated for 30 min at room temperature in the dark. The media was removed and the cells were then homogenized using DMSO. The cell lysates (50 μL) were then transferred to a 96 well plate and the fluorescence was measure on the plate reader (480 nm excitation, 530 nm emission).

BioD and PK of NPs in Pig.

American Landrace piglets (4 weeks old, 3 per group) were anesthetized using isofluorane. T-CO40-HDL-NPs, T-CO70-HDL-NPs (1.25 mg/kg with respect to total NP; 0.181 mg/kg for 40%-CO and 0.165 mg/kg for 70%-CO with respect to Cd), and saline were administered via intravenous catheter placed into an ear vein. Blood samples were collected in heparinized tubes at 0, 2, 4, 6, 8, and 24 h post-injection and stored at 4° C. until further use. Blood samples were centrifuged at 2000 rpms for 20 min at 4° C. in order to isolate plasma. The percentage of QD from NPs was calculated by taking into consideration that blood constitutes 3.5% of body weight and plasma constitutes 55% of blood volume for pigs. The amount of Cd from the QD was calculated in the blood plasma by ICP-MS. 24 h post-injection, piglets were anesthetized using 5% isofluorane with oxygen and then euthanized via CO2 inhalation. For the bioD studies, the heart, aorta, lungs, liver, kidneys, and spleen were isolated and stored at −80° C. until further use. Portions of the organs isolated were dissolved using concentrated nitric acid at 50° C. with gentle shaking. The overall bioD was calculated by analyzing the amount of Cd in each organ by ICP-MS. The heart and aorta were also imaged by IVIS using Cy5.5 emission and 500 nm excitation with an exposure time of 1 s. The calculations for AUC, Cmax, Tmax, and CL (t=0) were performed in the GraphPad Prism software. PK parameters were determined by fitting the data using a one compartmental model equation.

Enzyme-Linked Immunosorbent Assay (ELISA) on Pig Serum Samples.

Cytokines IL-6, IL-10, and TNF-α levels were measured after each time point collected according to manufacturer's protocol. All chemicals and standard solutions were warmed to room temperature before use. To the capture antibody pre-coated strips, the assay diluent (50 μL) was added. To this, standard or serum samples (50 μL) were added. The plate was then sealed with the adhesive strip provided and incubated for 2 h at room temperature on an orbital shaker. The wells were then aspirated and washed 5 times with the wash buffer (˜300 μL each wash). The freshly washed plates were then incubated with either IL-6, IL-10, or TNF-α conjugates (100 μL). Once again, they were sealed with the provided adhesive strips for 2 h at room temperature on an orbital shaker. The solutions were aspirated and washed 5 times with the wash buffer. Then, the wells were incubated with the substrate solution (100 μL) for 30 min at room temperature in the dark. After 30 min, the reaction was stopped with the stop solution (100 μL) with gentle mixing to ensure a uniform solution. The plates were then read for absorbance using a plate reader at 450 nm.

Triglyceride Quantification on Pig Serum Samples.

The isolated plasma samples from each time point (50 μL) were incubated with AdipoRed (5 μL) for 20 min at room temperature in a 96 well plate. The plate was then read for fluorescence with an excitation wavelength of 485 nm and an emission wavelength of 572 nm.

Cholesterol Quantification on Pig Serum Samples.

Serum cholesterol concentration was quantified by AmplexRed cholesterol quantification kit. The isolated plasma samples from each time point (50 μL) were incubated with a working reagent solution composed of AmplexRed (300 μM), HRP (0.2 U/mL), cholesterol oxidase (2 U/mL), and cholesterol esterase (0.2 U/mL) for 30 min at 37° C. in the dark. The fluorescence was measured using a plate reader at an excitation of 560 nm and an emission of 590 nm. RFUs were converted to cholesterol concentration using a standard curve of a cholesterol reference standard.

Statistics.

All data were expressed as mean±S.D (standard deviation). Statistical analysis were performed using GraphPad Prism® software v. 5.00. Comparisons between two values were performed using an unpaired Student t test. A one-way ANOVA with a post-hoc Tukey test was used to identify significant differences among the groups.

Supplemental Information

Materials and Instrumentations.

All chemicals were received and used without further purification unless otherwise noted. PLGA-COOH of inherent viscosity of 0.15-0.25 dL/g was purchased from Lactel. Cholesteryl oleate (CO) and nitric acid were purchased from Sigma-Aldrich. PerkinElmer solvable (Product number: 6NE9100) was purchased from Perkin Elmer. NBD-cholesterol, QDot 705 ITK amino PEG-QDs, and AmplexRed cholesterol quantification kit were purchased from Invitrogen. The apoA-I mimetic peptide L-4F peptide Ac-FAEKFKEAVKDYFAKFWD-COOH (SEQ ID NO:1) was custom synthesized by RS Synthesis and characterized by MALDI and HPLC. Carboxylic acid 1,2-distearoylsn-glycero-3-phosphoethanolamine-Ntcarboxy(polyethylene glycol)-2000] (DSPE-PEG-COOH) was purchased from Avanti Polar Lipids, Inc. Stearyl triphenylphosphonium bromide (Stearyl-TPP) and PLGA-PEG-QDs were synthesized according to methods previously described by Marrache, S. & Dhar, S. Biodegradable synthetic high-density lipoprotein nanoparticles for atherosclerosis. Proc Natl Acad Sci USA 110, 9445-9450 (2013). Polyvinyl alcohol (PVA) (86-89% hydrolyzed) of low molecular weight (average molecular weight of 10,000 to 26,000) was purchased from Alfa Aesar. AdipoRed was purchased from Lonza. Ultrapure lipopolysaccharide (LPS) from E. coli was purchased from InvivoGen. Native HDL from human plasma (BT-914) was purchased from Biomedical Technologies. Lipoprotein deficient fetal bovine serum (FBS) (RP-056) and modified human lipoprotein oxidized LDL (RP-047) were obtained from Intracell. Pig study cytokines were measured on a porcine Quantikine enzyme-linked immunosorbent assay (ELISA) kit from R&D systems. Reactive oxygen species (ROS) was measured using OxiSelect™ intracellular ROS assay kit.

Distilled water was purified by passage through a Millipore Milli-Q Biocel water purification system (18.2 MS2) containing a 0.22 μm filter. Cells were counted using Countess® Automated cell counter procured from Invitrogen. Dynamic light scattering (DLS) measurements were carried out using a Malvern Zetasizer Nano ZS system. Optical measurements were carried out on a NanoDrop 2000 spectrophotometer. Transmission electron microscopy (TEM) images were acquired using a Philips/FEI Technai 20 microscope. Inductively coupled plasma mass spectrometry (ICP-MS) studies were performed on a VG PlasmaQuad 3 ICP mass spectrometer. Plate reader analyses were performed on a Bio-Tek Synergy HT microplate reader. Anti-oxidative stress assays were carried out using a Seahorse XF24 analyzer (Seahorse Biosciences, North Billerica, Mass., USA). Fluorescence imaging of heart and aorta samples was carried out on a Xenogen IVIS® Lumina system.

Cell Line and Cell Culture.

Mouse macrophage RAW 264.7 cells were procured from American type culture collection (ATCC). RAW 264.7 cells were grown in Roswell Park Memorial Institute (RPMI) medium supplemented with L-glutamine, HEPES buffer, and sodium pyruvate at 37° C. in 5% CO2. Cells were passed every 2-3 days and restarted from frozen stocks upon reaching pass 20.

Synthesis of Stearyl-TPP.

Stearyl-TPP was synthesized and characterized by following methods previously reported by Marrache, S. & Dhar, S. Biodegradable synthetic high-density lipoprotein nanoparticles for atherosclerosis. Proc Natl Acad Sci USA 110, 9445-9450 (2013).

TABLE S1
Characterization of T-HDL-NPP library from three independent experiments

| | | Zeta | Polydispersity | | |
|---|---|---|---|---|---|
| | Z_{Average} | Potential | index | % CO | % EE |
| | (nm) | (mV) | (PDI) | Loading | of CO |
| T-CO₁₀-HDL-NP | 222.0 ± 41.5 | 49.9 ± 8.3 | 0.16 ± 0.03 | 5.2 ± 0.13 | 49.4 ± 1.2 |
| T-CO₂₀-HDL-NP | 166.4 ± 0.4 | 49.7 ± 9.6 | 0.19 ± 0.09 | 9.4 ± 2.19 | 41.8 ± 10.6 |
| T-CO₃₀-HDL-NP | 157.6 ± 2.3 | 55.5 ± 10.6 | 0.15 ± 0.07 | 12.7 ± 2.9 | 34.2 ± 8.7 |
| T-CO₄₀-HDL-NP | 151.6 ± 0.8 | 51.2 ± 6.9 | 0.12 ± 0.04 | 17.4 ± 405 | 31.9 ± 9.7 |
| T-CO₅₀-HDL-NP | 145.4 ± 0.7 | 55.2 ± 3.6 | 0.15 ± 0.02 | 22.3 ± 4.7 | 29.0 ± 7.5 |
| T-CO₆₀-HDL-NP | 143.4 ± 2.7 | 53.1 ± 8.5 | 0.14 ± 0.02 | 27.0 ± 5.1 | 24.9 ± 6.2 |
| T-CO₇₀-HDL-NP | 138.6 ± 4.0 | 49.9 ± 6.6 | 0.11 ± 0.01 | 34.2 ± 5.6 | 22.6 ± 5.4 |
| T-CO₈₀-HDL-NP | 131.3 ± 5.5 | 45.3 ± 14.5 | 0.13 ± 0.01 | 45.0 ± 5.9 | 20.8 ± 4.7 |
| T-CO₉₀-HDL-NP | 166.0 ± 22.9 | 46.5 ± 12.9 | 0.15 ± 0.02 | 69.8 ± 4.0 | 26.0 ± 4.6 |

TABLE S2
Characterization of T-HDL-NPP library from three independent experiments

| | | Zeta | Polydispersity | | |
|---|---|---|---|---|---|
| | Z_{Average} | Potential | index | % CO | % EE |
| | (nm) | (mV) | (PDI) | Loading | of CO |
| T-CO₁₀-HDL-NP | 129.3 ± 15.1 | −32.4 ± 2.4 | 0.17 ± 0.05 | 1.5 ± 0.1 | 13 ± 0.8 |
| T-CO₂₀-HDL-NP | 132.8 ± 19.5 | −29.9 ± 8.33 | 0.17 ± 0.07 | 2.8 ± 0.2 | 11.3 ± 0.7 |
| T-CO₃₀-HDL-NP | 138.1 ± 23.8 | −31.6 ± 6.9 | 0.19 ± 0.08 | 6.3 ± 0.5 | 14.7 ± 1.2 |
| T-CO₄₀-HDL-NP | 129.1 ± 6.9 | −35.2 ± 5.7 | 0.18 ± 0.04 | 7.9 ± 0.4 | 12 ± 0.6 |
| T-CO₅₀-HDL-NP | 140.9 ± 11.7 | −32.9 ± 7.6 | 0.22 ± 0.11 | 9.9 ± 0.2 | 9.9 ± 0.2 |
| T-CO₆₀-HDL-NP | 123.9 ± 18.6 | −33.8 ± 5.5 | 0.19 ± 0.03 | 15.2 ± 0.7 | 10.1 ± 0.5 |
| T-CO₇₀-HDL-NP | 123.5 ± 25.4 | −33.1 ± 4.8 | 0.22 ± 0.06 | 18.6 ± 0.5 | 7.9 ± 0.2 |
| T-CO₈₀-HDL-NP | 128.5 ± 19.3 | −30.2 ± 4.9 | 0.21 ± 0.06 | 24.8 ± 0.3 | 6.2 ± 0.1 |
| T-CO₉₀-HDL-NP | 133.9 ± 12.3 | −28.6 ± 6.9 | 0.22 ± 0.05 | 47.3 ± 0.7 | 5.3 ± 0.1 |

Table X below illustrates various ratios of cholesterol oleate (CO) to polymer core (PLGA-COOH/PLGA-COOCH₃); CO to ApoA1, ApoA1 to polymer core, and CO to HDL (everything together) of nanoparticles that may formed in accordance with the teachings presented herein.

| | % PLGA- | CO:PLGA- |
|---|---|---|
| | COOH/% PLGA- | COOH/PLGA- |
| % CO | COOCH₃ | COOCH₃ |
| 10 | 90 | 1:9 |
| 20 | 80 | 1:4 |
| 30 | 70 | 3:7 |
| 40 | 60 | 2:3 |
| 50 | 50 | 1:1 |
| 60 | 40 | 3:2 |
| 70 | 30 | 7:3 |
| 80 | 20 | 4:1 |
| 90 | 10 | 9:1 |

| % CO wrt PLGA- | % ApoAI wrt PLGA- | | |
|---|---|---|---|
| COOH/PLGA- | COOH/PLGA- | | |
| COOCH₃ | COOCH₃ | CO:ApoAI | CO:HDL |
| 10 | 3.33 | 2:1 | 1:2.75 |
| | 6.67 | 1:1 | 1:2.8 |
| | 10 | 1:1.5 | 1:2.85 |
| | 13.32 | 1:2 | 1:2.9 |
| | 16.65 | 1:2.5 | 1:2.95 |
| | 20 | 1:3 | 1:3 |
| | 23.31 | 1:3.5 | 1:3.05 |
| | 26.64 | 1:4 | 1:3.1 |
| | 30 | 1:4.5 | 1:3.15 |
| | 33.33 | 1:5 | 1:3.2 |
| 20 | 3.33 | 4:1 | 1:2.85 |
| | 6.67 | 2:1 | 1:2.9 |
| | 10 | 3:4 | 1:2.95 |
| | 13.32 | 1:1 | 1:3 |
| | 16.65 | 4:5 | 1:3.05 |
| | 20 | 2:3 | 1:3.1 |
| | 23.31 | 4:7 | 1:3.15 |
| | 26.64 | 1:2 | 1:3.2 |
| | 30 | 4:9 | 1:3.25 |
| | 33.33 | 2:5 | 1:3.3 |
| 30 | 3.33 | 6:1 | 1:2.95 |
| | 6.67 | 3:1 | 1:3 |
| | 10 | 2:1 | 1:3.05 |
| | 13.32 | 3:2 | 1:3.1 |
| | 16.65 | 3:2.5 | 1:3.15 |
| | 20 | 1:1 | 1:3.2 |
| | 23.31 | 3:3.5 | 1:3.25 |
| | 26.64 | 3:4 | 1:3.3 |
| | 30 | 3:4.5 | 1:3.35 |
| | 33.33 | 3:5 | 1:3.4 |
| 40 | 3.33 | 8:1 | 1:3.05 |
| | 6.67 | 4:1 | 1:3.1 |
| | 10 | 4:1.5 | 1:3.15 |
| | 13.32 | 2:1 | 1:3.2 |
| | 16.65 | 4:2.5 | 1:3.25 |
| | 20 | 4:3 | 1:3.3 |
| | 23.31 | 4:3.5 | 1:3.35 |
| | 26.64 | 1:1 | 1:3.4 |
| | 30 | 4:4.5 | 1:3.45 |
| | 33.33 | 4:5 | 1:3.5 |
| 50 | 3.33 | 10:1 | 1:3.15 |
| | 6.67 | 5:1 | 1:3.2 |
| | 10 | 5:1.5 | 1:3.25 |
| | 13.32 | 5:2 | 1:3.3 |
| | 16.65 | 2:1 | 1:3.35 |
| | 20 | 5:3 | 1:3.4 |
| | 23.31 | 5:3.5 | 1:3.45 |
| | 26.64 | 5:4 | 1:3.5 |
| | 30 | 5:4.5 | 1:3.55 |
| | 33.33 | 1:1 | 1:3.6 |
| 60 | 3.33 | 12:1 | 1:3.25 |
| | 6.67 | 6:1 | 1:3.3 |
| | 10 | 4:1 | 1:3.35 |
| | 13.32 | 3:1 | 1:3.4 |
| | 16.65 | 6:2.5 | 1:3.45 |
| | 20 | 2:1 | 1:3.5 |
| | 23.31 | 6:3.5 | 1:3.55 |
| | 26.64 | 3:2 | 1:3.6 |
| | 30 | 6:4.5 | 1:3.65 |
| | 33.33 | 6:5 | 1:3.7 |
| 70 | 3.33 | 14:1 | 1:3.35 |
| | 6.67 | 7:1 | 1:3.4 |
| | 10 | 7:1.5 | 1:3.45 |
| | 13.32 | 3.5:1 | 1:3.5 |
| | 16.65 | 7:2.5 | 1:3.55 |
| | 20 | 7:3 | 1:3.6 |
| | 23.31 | 2:1 | 1:3.65 |
| | 26.64 | 7:4 | 1:3.7 |
| | 30 | 7:4.5 | 1:3.75 |
| | 33.33 | 7:5 | 1:3.8 |
| 80 | 3.33 | 16:1 | 1:3.45 |
| | 6.67 | 8:1 | 1:3.5 |
| | 10 | 8:1.5 | 1:3.55 |
| | 13.32 | 4:1 | 1:3.6 |
| | 16.65 | 8:2.5 | 1:3.65 |
| | 20 | 8:3 | 1:3.7 |
| | 23.31 | 8:3.5 | 1:3.75 |
| | 26.64 | 2:1 | 1:3.8 |
| | 30 | 8:4.5 | 1:3.85 |
| | 33.33 | 8:5 | 1:3.9 |
| 90 | 3.33 | 18:1 | 1:3.55 |
| | 6.67 | 9:1 | 1:3.6 |
| | 10 | 9:1.5 | 1:3.65 |
| | 13.32 | 4.5:1 | 1:3.7 |
| | 16.65 | 9:2.5 | 1:3.75 |
| | 20 | 3:1 | 1:3.8 |
| | 23.31 | 9:3.5 | 1:3.85 |
| | 26.64 | 9:4 | 1:3.9 |
| | 30 | 2:1 | 1:3.95 |
| | 33.33 | 9:5 | 1:4 |

The inventors have found that, in general, a lower percentage of CO allows the nanoparticles to accommodate more cholesterol in the core.

Thus, embodiments of NANOPARTICLES FOR LIPID HOMEOSTASIS are disclosed. One skilled in the art will appreciate that the nanoparticles and methods described herein can be practiced with embodiments other than those disclosed. The disclosed embodiments are presented for purposes of illustration and not limitation.

All journal articles, patents and published patent applications referred to herein are hereby incorporated herein by reference in their respective entireties to the extent that they do not conflict with the present disclosure.

1. A nanoparticle, comprising:
a polymeric core;
a high density lipoprotein (HDL) component, wherein the ratio by weight of the HDL component to the one or more polymers forming the core is in a range from about 1:9 to about 9:1; and
a mitochondria targeting moiety attached to the core.

2. A nanoparticle according to claim 1, wherein the ratio by weight of the HDL component to the one or more polymers forming the core is in a range from about 75:25 or less.

3. A nanoparticle according to claim 1, wherein the ratio by weight of the HDL component to the one or more polymers forming the core is in a range from about 7:3 or less.

4. A nanoparticle according to claim 1, wherein the ratio by weight of the HDL component to the one or more polymers forming the core is in a range from about 1:4 to about 3:2.

5. A nanoparticle according to claim 1, wherein the ratio by weight of the HDL component to the one or more polymers forming the core is about 2:3.

6. A nanoparticle according to claim 1, wherein the core is formed, at least in part, from poly(lactic-co-glycolic) acid (PLGA).

7. A nanoparticle according to claim 1, wherein the core consists essentially of poly(lactic-co-glycolic) acid (PLGA).

8. A nanoparticle according to claim 1, wherein the HDL component comprises a cholesterol moiety.

9. A nanoparticle according to claim 1, wherein the HDL component comprises cholesteryl oleate.

10. A nanoparticle according to claim 1, further comprising an apoA-I mimetic polypeptide.

11. A nanoparticle according to claim 10, wherein the apoA-1 mimetic polypeptide comprises an L-4F polypeptide.

12. A nanoparticle according to claim 11, wherein the apoA-1 mimetic polypeptide comprises an amino acid sequence of FAEKFKEAVKDYFAKFWD.

13. A nanoparticle according to claim 1, further comprising a phospholipid monolayer attached to the core.

14. A nanoparticle according to claim 13, wherein the phospholipid monolayer layer is formed at least in part from a compound comprising a distearoyl-snglycero-3-phosphoethanolamine (DSPE) moiety or a derivative thereof or cardiolipin.

15. A nanoparticle according to claim 1, wherein the mitochondrial matrix targeting moiety comprises a moiety selected from the group consisting of a triphenyl phosophonium (TPP) moiety, a Szeto-Shiller peptide, and a rhodamine cation.

16. A nanoparticle according to claim 1, wherein the mitochondrial matrix targeting moiety comprises a triphenyl phosophonium (TPP) moiety or a derivative thereof.

17. A method for maintaining lipid homeostasis in a subject in need thereof, comprising:
administering to the subject an effective amount of a nanoparticle according to claim 1.

18. Use of a nanoparticle according to claim 1 for maintaining lipid homeostasis in a subject in need thereof.


SEQUENCE LISTING (2 sequences)

SEQ ID NO: 1 - protein, 18 aa, Artificial Sequence
  Synthetic polypeptide
FAEKFKEAVKDYFAKFWD

SEQ ID NO: 2 - protein, 7 aa, Artificial Sequence
  Synthetic polypeptide
GPLGVRG